(19) United States
(12) Patent Application Publication
(10) Pub. No.: US 2017/0128006 A1
(43) Pub. Date: May 11, 2017

(54) ELECTRONIC DEVICE FOR PROVIDING HEALTH INFORMATION AND OPERATION METHOD THEREOF

(71) Applicant: Samsung Electronics Co., Ltd., Gyeonggi-do (KR)

(72) Inventors: Hye-Jung SEO, Gyeonggi-do (KR); Sook-Jin KIM, Gyeonggi-do (KR); Won-Suk CHOI, Seoul (KR)

(21) Appl. No.: 15/347,144

(22) Filed: Nov. 9, 2016

(30) Foreign Application Priority Data
Nov. 11, 2015 (KR) 10-2015-0158286

(51) Int. Cl. A61B 5/00; A61B 10/00; A61B 5/0205; A61B 5/145

(52) U.S. Cl. CPC A61B 5/4845; A61B 5/14517; A61B 10/0064; A61B 5/0205; A61B 5/14532; A61B 5/4866; A61B 5/0002; A61B 5/7246; A61B 5/742; A61B 2010/0009; A61B 2010/0067; A61B 2010/0087; A61B 5/021

(57) ABSTRACT

An electronic device and method are disclosed herein. The electronic device includes a sensor, and a processor. The processor may implement the method, including acquiring, by the sensor, first biometric information of a user, determining alcohol information for determining a presence of of alcohol in the user from the first biometric information, determining second biometric information of the user related to the alcohol information, and generating health information to be displayed the user or transmitted to another electronic device based on the second biometric information.

### CLAIM OF PRIORITY

This application claims priority under 35 U.S.C. §119(a) to Korean Application Serial No. 10-2015-0158286, which was filed in the Korean Intellectual Property Office on Nov. 11, 2015, the entire content of which is hereby incorporated by reference.

### TECHNICAL FIELD

The present disclosure relates to a providing health information and, more particularly, to electronically providing health information to a user based on biometric information acquired through a sensor and an operation method thereof.

### BACKGROUND

A recent mobile electronic device may include a sensor which can measure a biometric signal from a body. The sensor can measure biometric signals, such as a heart rate or a blood alcohol concentration. The mobile electronic device may provide information relating to a user's body via processing and analysis of the measured biometric signal. The user may be informed as to his/her own body status by the information generated by the mobile electronic device.

### SUMMARY

A use frequency and use effect of a portable biometric information measurement device may vary significantly depending on a user operation. A measurement device that simply shows a value and acquires data may be limited in terms of providing customized health information or a health guide.

According to various embodiments of the present disclosure, an electronic device for acquiring alcohol information through user's biometric information and providing health information to a user based on the alcohol information and a method of operating the same are provided.

In accordance with an aspect of the present disclosure, an electronic device is provided. The electronic device includes: a sensor and a processor. The processor is configured to acquire, by the sensor, first biometric information of a user, determine alcohol information for determining a presence of of alcohol in the user from the first biometric information, determine second biometric information of the user related to the alcohol information, and generate health information to be displayed the user or transmitted to another electronic device based on the second biometric information.

In accordance with another aspect of the present disclosure, a method of operating an electronic device including a sensor is provided. The method includes: acquiring, by the sensor, first biometric information of a user, determining alcohol information for determining a presence of alcohol in the user from the first biometric information, determining second biometric information of the user related to the alcohol information, and generating health information to be displayed to the user or transmitted to another electronic device based on the second biometric information.

In accordance with another aspect of the present disclosure, a non-transitory storage medium storing instructions executable by at least one processor to perform at least one operation is disclosed. The at least one operation may include acquiring, by the sensor, first biometric information of a user through, determining alcohol information for determining a presence of alcohol in the user from the first biometric information, determining second biometric information of the user related to the alcohol information, and generating health information to be displayed to the user or transmitted to another electronic device based on the second biometric information.

An electronic device according to various embodiments of the present disclosure can acquire alcohol information through user's biometric information and provide more accurate and various health information to a user based on the acquired alcohol information.

### BRIEF DESCRIPTION OF THE DRAWINGS

The above and other aspects and features of the present disclosure will be more apparent from the following detailed description taken in conjunction with the accompanying drawings, in which:

FIG. 1 is a block diagram illustrating an electronic device and a network according to various embodiments of the present disclosure;

FIG. 2 is a block diagram illustrating an electronic device according to various embodiments;

FIG. 3 is a block diagram illustrating a program module according to various embodiments;

FIG. 4 is a schematic block diagram illustrating an electronic system according to an embodiment of the present disclosure;

FIG. 5 is a schematic block diagram illustrating an electronic system according to another embodiment of the present disclosure;

FIG. 6 is a data flow illustrating an operation method of an electronic device according to various embodiments of the present disclosure;

FIG. 7 is a data flow illustrating an operation method of an electronic device according to various embodiments of the present disclosure;

FIG. 8 illustrates an interface for health information provided by the electronic device according to an embodiment of the present disclosure;

FIG. 9 illustrates an interface for health information provided by the electronic device according to another embodiment of the present disclosure;

FIG. 10 is a flowchart illustrating an operation method of the electronic device according to various embodiments of the present disclosure;

FIG. 11 is a flowchart illustrating an operation method of the electronic device according to various embodiments of the present disclosure;

FIG. 12 is a flowchart illustrating an operation method of the electronic device according to various embodiments of the present disclosure;

FIG. 13 is a flowchart illustrating an operation method of the electronic device according to various embodiments of the present disclosure;

FIG. 14A and FIG. 14B are graphs illustrating health information provided by the electronic device according to various embodiments of the present disclosure;

FIG. 15 is a graph illustrating health information provided by the electronic device according to various embodiments of the present disclosure;

FIG. 16 is a graph illustrating health information provided by the electronic device according to various embodiments of the present disclosure; and

FIG. 17 is a graph illustrating a method of measuring health information by the electronic device according to an embodiment of the present disclosure.

### DETAILED DESCRIPTION

Hereinafter, various embodiments of the present disclosure will be described with reference to the accompanying drawings. However, it should be understood that there is no intent to limit the present disclosure to the particular forms disclosed herein; rather, the present disclosure should be construed to cover various modifications, equivalents, and/or alternatives of embodiments of the present disclosure. In describing the drawings, similar reference numerals may be used to designate similar constituent elements.

As used herein, the expression “have”, “may have”, “include”, or “may include” refers to the existence of a corresponding feature (e.g., numeral, function, operation, or constituent element such as component), and does not exclude one or more additional features.

In the present disclosure, the expression “A or B”, “at least one of A or/and B”, or “one or more of A or/and B” may include all possible combinations of the items listed. For example, the expression “A or B”, “at least one of A and B”, or “at least one of A or B” refers to all of (1) including at least one A, (2) including at least one B, or (3) including all of at least one A and at least one B.

The expression “a first”, “a second”, “the first”, or “the second” used in various embodiments of the present disclosure may modify various components regardless of the order and/or the importance but does not limit the corresponding components. For example, a first element may be termed a second element, and similarly, a second element may be termed a first element without departing from the present disclosure. For example, a first element may be termed a second element, and similarly, a second element may be termed a first element without departing from the present disclosure.

It should be understood that when an element (e.g., first element) is referred to as being (operatively or communicatively) “connected,” or “coupled,” to another element (e.g., second element), it may be directly connected or coupled directly to the other element or any other element (e.g., third element) may be interposer between them. Conversely, when it is mentioned that one element (e.g., a first element) is “directly coupled” or “directly connected” to another element (e.g., a second element), it may be construed that yet another element does not exist between the one element and the another element.

The expression “configured to” used in the present disclosure may be exchanged with, for example, “suitable for”, “having the capacity to”, “designed to”, “adapted to”, “made to”, or “capable of” according to the situation. The term “configured to” may not necessarily imply “specifically designed to” in hardware. Alternatively, in some situations, the expression “device configured to” may mean that the device, together with other devices or components, “is able to”. For example, the phrase “processor adapted (or configured) to perform A, B, and C” may mean a dedicated processor (e.g., embedded processor) for performing the corresponding operations or a generic-purpose processor (e.g., central processing unit (CPU) or application processor (AP)) that can perform the corresponding operations by executing one or more software programs stored in a memory device.

The terms used herein are merely for the purpose of describing particular embodiments and may not be intended to limit the other embodiments. A singular expression may include a plural expression unless they are definitely different in a context. Unless defined otherwise, all terms used herein, including technical and scientific terms, have the same meaning as those commonly understood by a person skilled in the art to which the present disclosure pertains. Such terms as those defined in a generally used dictionary may be interpreted to have the meanings equal to the contextual meanings in the relevant field of art, and are not to be interpreted to have ideal or excessively formal meanings unless clearly defined in the present disclosure. In some cases, even the term defined in the present disclosure should not be interpreted to exclude embodiments of the present disclosure.

An electronic device according to various embodiments of the present disclosure may include at least one of, for example, a smart phone, a tablet Personal Computer (PC), a mobile phone, a video phone, an electronic book reader (e-book reader), a desktop PC, a laptop PC, a netbook computer, a workstation, a server, a Personal Digital Assistant (PDA), a Portable Multimedia Player (PMP), a MPEG-1 audio layer-3 (MP3) player, a mobile medical device, a camera, and a wearable device. According to various embodiments, the wearable device may include at least one of an accessory type (e.g., a watch, a ring, a bracelet, an anklet, a necklace, a glasses, a contact lens, or a Head-Mounted Device (HMD)), a fabric or clothing integrated type (e.g., an electronic clothing), a body-mounted type (e.g., a skin pad, or tattoo), and a bio-implantable type (e.g., an implantable circuit).

According to some embodiments, the electronic device may be a home appliance. The home appliance may include at least one of, for example, a television, a Digital Video Disk (DVD) player, an audio, a refrigerator, an air conditioner, a vacuum cleaner, an oven, a microwave oven, a washing machine, an air cleaner, a set-top box, a home automation control panel, a security control panel, a TV box (e.g., Samsung HomeSync™, Apple TV™, or Google TV™), a game console (e.g., Xbox™ and PlayStation™), an electronic dictionary, an electronic key, a camcorder, and an electronic photo frame.

According to another embodiment, the electronic device may include at least one of various medical devices (e.g., various portable medical measuring devices (a blood glucose monitoring device, a heart rate monitoring device, a blood pressure measuring device, a body temperature measuring device, etc.), a Magnetic Resonance Angiography (MRA), a Magnetic Resonance Imaging (MRI), a Computed Tomography (CT) machine, and an ultrasonic machine), a navigation device, a Global Positioning System (GPS) receiver, an Event Data Recorder (EDR), a Flight Data Recorder (FDR), a Vehicle Infotainment Devices, an electronic devices for a ship (e.g., a navigation device for a ship, and a gyro-compass), avionics, security devices, an automotive head unit, a robot for home or industry, an automatic teller's machine (ATM) in banks, point of sales (POS) in a shop, or internet device of things (e.g., a light bulb, various sensors, electric or gas meter, a sprinkler device, a fire alarm, a thermostat, a streetlamp, a toaster, a sporting goods, a hot water tank, a heater, a boiler, etc.).

According to some embodiments, the electronic device may include at least one of a part of furniture or a building/structure, an electronic board, an electronic signature receiving device, a projector, and various kinds of measuring instruments (e.g., a water meter, an electric meter, a gas meter, and a radio wave meter). The electronic device according to various embodiments of the present disclosure may be a combination of one or more of the aforementioned various devices. The electronic device according to some embodiments of the present disclosure may be a flexible device. Further, the electronic device according to an embodiment of the present disclosure is not limited to the aforementioned devices, and may include a new electronic device according to the development of technology.

Hereinafter, an electronic device according to various embodiments of the present disclosure will be described with reference to the accompanying drawings. In the present disclosure, the term “user” may indicate a person using an electronic device or a device (e.g., an artificial intelligence electronic device) using an electronic device.

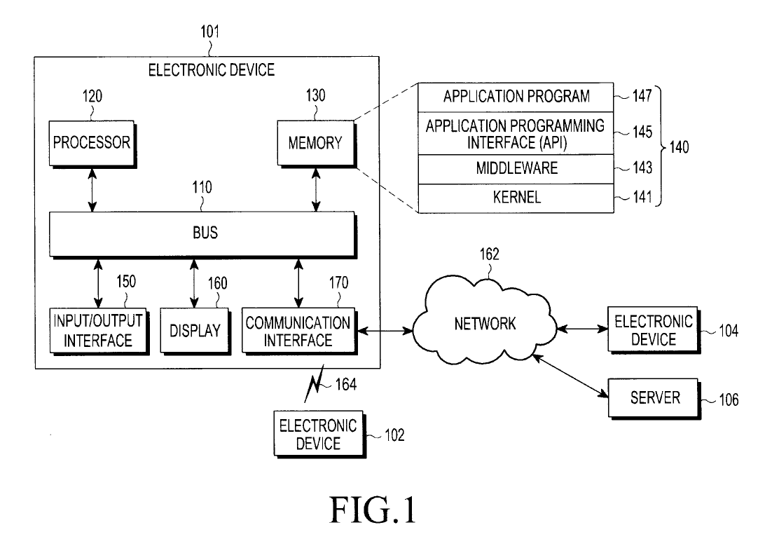

An electronic device 101 in a network environment 100 according to various embodiments will be described with reference to FIG. 1. The electronic device 101 may include a bus 110, a processor 120, a memory 130, an input/output interface 150, a display 160, and a communication interface 170. In some embodiments, the electronic device 101 may omit at least one of the above elements or may further include other elements.

The bus 110 may include, for example, a circuit which interconnects the components 110 to 170 and delivers communication (for example, a control message and/or data) between the components 110 to 170.

The processor 120 may include one or more of a Central Processing Unit (CPU), an Application Processor (AP), and a Communication Processor (CP). For example, the processor 120 may carry out operations or data processing related to control and/or communication of at least one other component of the electronic device 101.

The memory 130 may include a volatile memory and/or a non-volatile memory. The memory 130 may store, for example, instructions or data relevant to at least one other element of the electronic device 101. According to an embodiment, the memory 130 may store software and/or a program 140. For example, the program 140 may include a kernel 141, a middleware 143, an Application Programming Interface (API) 145, and/or application programs (or applications) 147. At least some of the kernel 141, the middleware 143, and the API 145 may be referred to as an Operating System (OS).

The kernel 141 may control or manage, for example, system resources (for example, the bus 110, the processor 120, and the memory 130) which are used to execute an operation or a function implemented in the other programs (for example, the middleware 143, the API 145, and the application programs 147). Furthermore, the kernel 141 may provide an interface through which the middleware 143, the API 145, or the application programs 147 may access the individual elements of the electronic device 101 to control or manage the system resources.

The middleware 143 may function as, for example, an intermediary for allowing the API 145 or the application programs 147 to communicate with the kernel 141 to exchange data.

In addition, the middleware 143 may process one or more task requests received from the application programs 147 according to priorities thereof. For example, the middleware 143 may assign priorities for using the system resources (for example, the bus 110, the processor 120, the memory 130, or the like) of the electronic device 101, to at least one of the application programs 147. For example, the middleware 143 may perform scheduling or load balancing on the one or more task requests by processing the one or more task requests according to the priorities assigned thereto.

The API 145 is an interface through which the application programs 147 control functions provided from the kernel 141 or the middleware 143, and may include, for example, at least one interface or function (for example, instruction) for file control, window control, image processing, or text control.

The input/output interface 150 may function as, for example, an interface that may transfer instructions or data input from a user or another external device to the other element(s) of the electronic device 101. Furthermore, the input/output interface 150 may output the instructions or data received from the other element(s) of the electronic device 101 to the user or another external device.

The display 160 may include, for example, a Liquid Crystal Display (LCD), a Light Emitting Diode (LED) display, an Organic Light Emitting Diode (OLED) display, a Micro Electro Mechanical System (MEMS) display, or an electronic paper display. The display 160, for example, may display various types of contents (for example, text, images, videos, icons, or symbols) for the user. The display 160 may include a touch screen and receive, for example, a touch, gesture, proximity, or hovering input using an electronic pen or the user's body part.

The communication interface 170, for example, may set communication between the electronic device 101 and an external device (for example, a first external electronic device 102, a second external electronic device 104, or a server 106). For example, the communication interface 170 may be connected to a network 162 through wireless or wired communication to communicate with the external device (for example, the second external electronic device 104 or the server 106).

The wireless communication may use at least one of, for example, Long Term Evolution (LTE), LTE-Advance (LTE-A), Code Division Multiple Access (CDMA), Wideband CDMA (WCDMA), Universal Mobile Telecommunications System (UMTS), WiBro (Wireless Broadband), and Global System for Mobile Communications (GSM), as a cellular communication protocol. In addition, the wireless communication may include, for example, short-range communication 164. The short-range communication 164 may be performed by using at least one of, for example, Wi-Fi, Bluetooth, Near Field Communication (NFC), and Global Navigation Satellite System (GNSS). The GNSS may include at least one of, for example, a Global Positioning System (GPS), a Global Navigation Satellite System (Glonass), a Beidou Navigation Satellite System (hereinafter referred to as “Beidou”), and a European Global Satellite-based Navigation System (Galileo), according to a use area, a bandwidth, or the like. Hereinafter, in the present disclosure, the “GPS” may be interchangeably used with the “GNSS”. The wired communication may include, for example, at least one of a Universal Serial Bus (USB), a High Definition Multimedia Interface (HDMI), Recommended Standard 232 (RS-232), and a Plain Old Telephone Service (POTS). The network 162 may include at least one of communication networks such as a computer network (for example, a LAN or a WAN), the Internet, and a telephone network.

Each of the first and second external electronic devices 102 and 104 may be of a type identical to or different from that of the electronic device 101. According to an embodiment, the server 106 may include a group of one or more servers. According to various embodiments, all or some of the operations executed by the electronic device 101 may be executed by another electronic device, a plurality of electronic devices (for example, the electronic devices 102 and 104), or the server 106. According to an embodiment, when the electronic device 101 has to perform a function or service automatically or in response to a request, the electronic device 101 may request another device (for example, the electronic device 102 or 104, or the server 106) to perform at least some functions relating thereto, instead of autonomously or additionally performing the function or service. Another electronic device (for example, the electronic device 102 or 104, or the server 106) may execute the requested functions or the additional functions, and may deliver a result of the execution to the electronic device 101. The electronic device 101 may provide the received result as it is or additionally process the received result and provide the requested functions or services. To achieve this, for example, cloud computing, distributed computing, or client-server computing technology may be used.

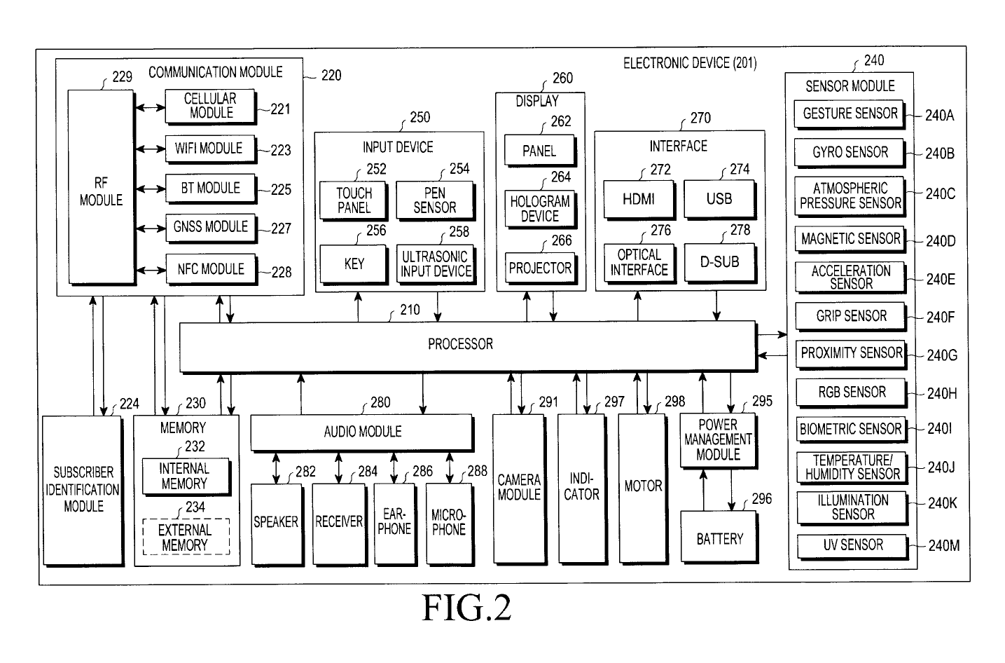

FIG. 2 is a block diagram illustrating an electronic device 201 according to various embodiments. The electronic device 201 may include, for example, the whole or part of the electronic device 101 illustrated in FIG. 1. The electronic device 201 may include at least one Application Processor (AP) 210, a communication module (e.g., circuit) 220, a Subscriber Identification Module (SIM) card 224, a memory 230, a sensor module 240, an input device 250, a display 260, an interface 270, an audio module 280, a camera module 291, a power management module 295, a battery 296, an indicator 297, and a motor 298.

The processor 210 may control multiple hardware or software elements connected to the processor 210 by running, for example, an Operation System (OS) or an application program, and may perform processing of and arithmetic operations on various data. The processor 210 may be implemented by, for example, a System on Chip (SoC). According to an embodiment, the processor 210 may further include a Graphic Processing Unit (GPU) and/or an image signal processor. The processor 210 may also include at least some (for example, a cellular module 221) of the elements illustrated in FIG. 2. The processor 210 may load, into a volatile memory, instructions or data received from at least one (for example, a non-volatile memory) of the other elements and may process the loaded instructions or data, and may store various data in a non-volatile memory.

The communication module 220 may have a configuration identical or similar to that of the communication interface 170 illustrated in FIG. 1. The communication circuit 220 may include, for example, a cellular module 221, a Wi-Fi module 223, a BT module 225, a GNSS module 227 (for example, a GPS module, a Glonass module, a Beidou module, or a Galileo module), an NFC module 228, and a Radio Frequency (RF) module 229.

The cellular module 221 may provide a voice call, an image call, a text message service, or an Internet service through, for example, a communication network. According to an embodiment, the cellular module 221 may distinguish between and authenticate electronic devices 201 within a communication network using a subscriber identification module (for example, the SIM card 224). According to an embodiment, the cellular module 221 may perform at least some of the functions that the AP 210 may provide. According to an embodiment, the cellular module 221 may include a Communication Processor (CP).

For example, each of the Wi-Fi module 223, the BT module 225, the GPS or GNSS module 227, and the NFC module 228 may include a processor for processing data transmitted/received through the corresponding module. In some embodiments, at least some (two or more) of the cellular module 221, the Wi-Fi module 223, the Bluetooth module 225, the GNSS module 227, and the NFC module 228 may be included in a single Integrated Chip (IC) or IC package.

The RF module 229 may transmit/receive, for example, a communication signal (for example, an RF signal). The RF module 229 may include, for example, a transceiver, a Power Amp Module (PAM), a frequency filter, a Low Noise Amplifier (LNA), or an antenna. According to another embodiment, at least one of the cellular module 221, the Wi-Fi module 223, the Bluetooth module 225, the GNSS module 227, and the NFC module 228 may transmit/receive an RF signal through a separate RF module.

The subscriber identification module 224 may include, for example, a card including a subscriber identity module and/or an embedded SIM, and may contain unique identification information (for example, an Integrated Circuit Card Identifier (ICCID)) or subscriber information (for example, an International Mobile Subscriber Identity (IMSI)).

The memory 230 (for example, the memory 130) may include, for example, an internal memory 232 or an external memory 234. The internal memory 232 may include at least one of, for example, a volatile memory (for example, a Dynamic Random Access Memory (DRAM), a Static RAM (SRAM), a Synchronous Dynamic RAM (SDRAM), and the like) and a non-volatile memory (for example, a One Time Programmable Read Only Memory (OTPROM), a Programmable ROM (PROM), an Erasable and Programmable ROM (EPROM), an Electrically Erasable and Programmable ROM (EEPROM), a flash memory (for example, a NAND flash memory or a NOR flash memory), a hard driver, or a Solid State Drive (SSD).

The external memory 234 may further include a flash drive, for example, a Compact Flash (CF), a Secure Digital (SD), a Micro Secure Digital (Micro-SD), a Mini Secure Digital (Mini-SD), an eXtreme Digital (xD), a Multi-Media Card (MMC), a memory stick, or the like. The external memory 234 may be functionally and/or physically connected to the electronic device 201 through various interfaces.

The sensor module 240 may measure a physical quantity or detect an operation state of the electronic device 201, and may convert the measured or detected information into an electrical signal. The sensor module 240 may include, for example, at least one of a gesture sensor 240A, a gyro sensor 240B, an atmospheric pressure sensor 240C, a magnetic sensor 240D, an acceleration sensor 240E, a grip sensor 240F, a proximity sensor 240G, a color sensor 240H (for example, a red, green, blue (RGB) sensor), a biometric sensor 240I, a temperature/humidity sensor 240J, a light sensor 240K, and a ultraviolet (UV) sensor 240M. Additionally or alternatively, the sensor module 240 may include, for example, an E-nose sensor, an electromyography (EMG) sensor, an electroencephalogram (EEG) sensor, an electrocardiogram (ECG) sensor, an Infrared (IR) sensor, an iris sensor, and/or a fingerprint sensor. The sensor module 240 may further include a control circuit for controlling one or more sensors included therein. In some embodiments, an electronic device 201 may further include a processor configured to control the sensor module 240 as a part of or separately from the processor 210, and may control the sensor module 240 while the processor 210 is in a sleep state.

The input device 250 may include, for example, a touch panel 252, a (digital) pen sensor 254, a key 256, and an ultrasonic input device or unit 258. The touch panel 252 may use at least one of, for example, a capacitive scheme, a resistive scheme, an infrared scheme, and an ultrasonic scheme. Further, the touch panel 252 may further include a control circuit. The touch panel 252 may further include a tactile layer and provide a tactile reaction to the user.

The (digital) pen sensor 254 may include, for example, a recognition sheet which is a part of the touch panel or is separated from the touch panel. The key 256 may include, for example, a physical button, an optical key or a keypad. The ultrasonic input device 258 may detect ultrasonic waves generated by an input tool through a microphone (for example, the microphone 288) and identify data corresponding to the detected ultrasonic waves.

The display 260 (for example, the display 160) may include a panel 262, a hologram device 264, or a projector 266. The panel 262 may include a configuration identical or similar to that of the display 160 illustrated in FIG. 1. The panel 262 may be implemented to be, for example, flexible, transparent, or wearable. The panel 262 and the touch panel 252 may be implemented as one module. The hologram device 264 may show a three dimensional (3D) image in the air by using an interference of light. The projector 266 may display an image by projecting light onto a screen. The screen may be located, for example, in the interior of or on the exterior of the electronic device 201. According to an embodiment, the display 260 may further include a control circuit for controlling the panel 262, the hologram device 264, or the projector 266.

The interface 270 may include, for example, a High-Definition Multimedia Interface (HDMI) 272, a Universal Serial Bus (USB) 274, an optical interface 276, or a D-subminiature (D-sub) 278. The interface 270 may be included in, for example, the communication interface 170 illustrated in FIG. 1. Additionally or alternatively, the interface 270 may include, for example, a Mobile High-definition Link (MHL) interface, a Secure Digital (SD) card/Multi-Media Card (MMC) interface, or an Infrared Data Association (IrDA) standard interface.

For example, the audio module 280 may bidirectionally convert between a sound and an electrical signal. At least some elements of the audio module 280 may be included in, for example, the input/output interface 150 illustrated in FIG. 1. The audio module 280 may process sound information which is input or output through, for example, a speaker 282, a receiver 284, earphones 286, the microphone 288 or the like.

The camera module 291 is a device which may photograph a still image and a dynamic image. According to an embodiment, the camera module 291 may include one or more image sensors (for example, a front sensor or a back sensor), a lens, an Image Signal Processor (ISP) or a flash (for example, LED or xenon lamp).

The power management module 295 may manage, for example, power of the electronic device 201. According to an embodiment, the power management module 295 may include a Power Management Integrated Circuit (PMIC), a charger Integrated Circuit (IC), or a battery or fuel gauge. The PMIC may use a wired and/or wireless charging method. Examples of the wireless charging method may include, for example, a magnetic resonance method, a magnetic induction method, an electromagnetic method, and the like. Additional circuits (for example, a coil loop, a resonance circuit, a rectifier, etc.) for wireless charging may be further included. The battery gauge may measure, for example, a residual quantity of the battery 296, and a voltage, a current, or a temperature during the charging. The battery 296 may include, for example, a rechargeable battery or a solar battery.

The indicator 297 may display a particular state, for example, a booting state, a message state, a charging state, or the like of the electronic device 201 or a part (for example, the processor 210) of the electronic device 201. The motor 298 may convert an electrical signal into mechanical vibration, and may generate vibration, a haptic effect, or the like. Although not illustrated, the electronic device 201 may include a processing unit (for example, a GPU) for supporting a mobile television (TV). The processing unit for supporting mobile TV may process, for example, media data according to a certain standard such as Digital Multimedia Broadcasting (DMB), Digital Video Broadcasting (DVB), or mediaFLO™.

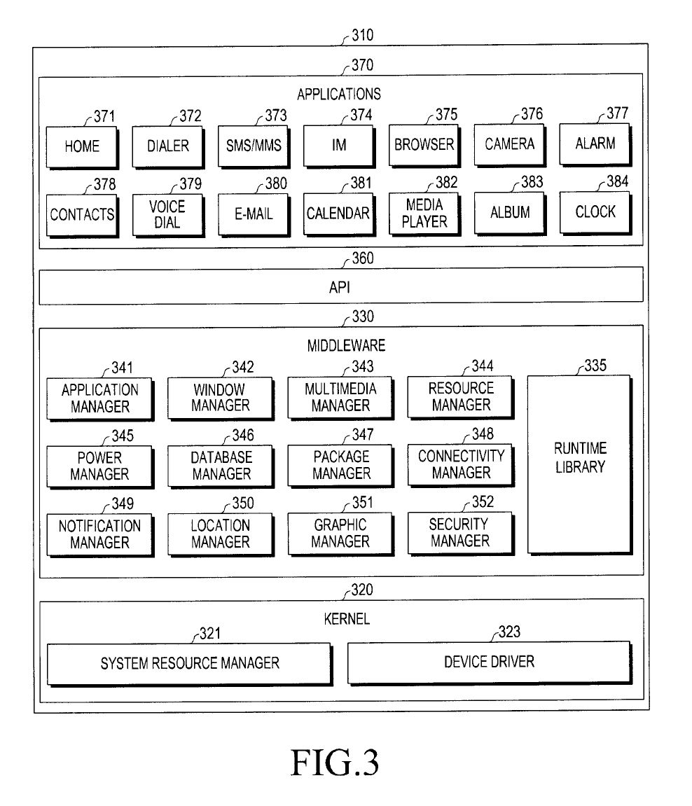

Each of the above-described component elements of hardware according to the present disclosure may be configured with one or more components, and the names of the corresponding component elements may vary based on the type of electronic device. The electronic device according to various embodiments of the present disclosure may include at least one of the aforementioned elements. Some elements may be omitted or other additional elements may be further included in the electronic device. Also, some of the hardware components according to various embodiments may be combined into one entity, which may perform functions identical to those of the relevant components before the combination. FIG. 3 is a block diagram illustrating a program module according to various embodiments. According to an embodiment, the program module 310 (for example, the program 140) may include an Operating System (OS) for controlling resources related to the electronic device (for example, the electronic device 101) and/or various applications (for example, the application programs 147) executed in the operating system. The operating system may be, for example, Android, iOS, Windows, Symbian, Tizen, Bada, or the like.

The program module 310 may include a kernel 320, middleware 330, an Application Programming Interface (API) 360, and/or applications 370. At least some of the program module 310 may be preloaded on the electronic device, or may be downloaded from an external electronic device (for example, the electronic device 102 or 104, or the server 106).

The kernel 320 (for example, the kernel 141) may include, for example, a system resource manager 321 and/or a device driver 323. The system resource manager 321 may control, assign, or collect system resources. According to an embodiment, the system resource manager 321 may include a process management unit, a memory management unit, or a file system management unit. The device driver 323 may include, for example, a display driver, a camera driver, a Bluetooth driver, a shared memory driver, a USB driver, a keypad driver, a Wi-Fi driver, an audio driver, or an Inter-Process Communication (IPC) driver.

The middleware 330 may provide a function utilized by the applications 370 in common or provide various functions to the applications 370 through the API 360 so that the applications 370 can efficiently use limited system resources within the electronic device. According to an embodiment, the middleware 330 (for example, the middleware 143) may include, for example, at least one of a runtime library 335, an application manager 341, a window manager 342, a multimedia manager 343, a resource manager 344, a power manager 345, a database manager 346, a package manager 347, a connectivity manager 348, a notification manager 349, a location manager 350, a graphic manager 351, and a security manager 352.

The runtime library 335 may include, for example, a library module that a compiler uses in order to add new functions through a programming language while the applications 370 are executed. The runtime library 335 may perform input/output management, memory management, or a function for an arithmetic function.

The application manager 341 may manage, for example, a life cycle of at least one of the applications 370. The window manager 342 may manage Graphical User Interface (GUI) resources used on a screen. The multimedia manager 343 may determine a format utilized to reproduce various media files, and may encode or decode a media file by using a coder/decoder (codec) appropriate for the corresponding format. The resource manager 344 may manage resources, such as a source code, a memory, a storage space, and the like of at least one of the applications 370.

The power manager 345 may operate together with, for example, a Basic Input/Output System (BIOS) to manage a battery or power and may provide power information utilized for the operation of the electronic device. The database manager 346 may generate, search for, and/or change a database to be used by at least one of the applications 370. The package manager 347 may manage the installation or update of an application distributed in the form of a package file.

The connectivity manager 348 may manage a wireless connection such as, for example, Wi-Fi or Bluetooth. The notification manager 349 may display or notify of an event, such as an arrival message, an appointment, proximity notification, and the like, in such a manner of not disturbing a user. The location manager 350 may manage location information of the electronic device. The graphic manager 351 may manage a graphic effect to be provided to a user and a user interface relating to the graphic effect. The security manager 352 may provide all security functions utilized for system security or user authentication. According to an embodiment, when the electronic device (for example, the electronic device 101) has a telephone call function, the middleware 330 may further include a telephony manager for managing a voice call function or a video call function of the electronic device.

The middleware 330 may include a middleware module that forms a combination of various functions of the above-described components. The middleware 330 may provide modules specialized according to types of operating systems in order to provide differentiated functions. Furthermore, the middleware 330 may dynamically remove some of the existing elements, or may add new elements.

The API 360 (for example, the API 145), which is a set of API programming functions, may be provided in a different configuration for each operating system. For example, in the case of Android or iOS, one API set may be provided for each platform, and in the case of Tizen, two or more API sets may be provided for each platform.

The applications 370 (for example, the application programs 147) may include, for example, one or more applications that can perform functions, such as home 371, dialer 372, SMS/MMS 373, Instant Message (IM) 374, browser 375, camera 376, alarm 377, contacts 378, voice dial 379, e-mail 380, calendar 381, media player 382, album 383, clock 384, health care (for example, measure exercise quantity or blood sugar), or environment information (for example, atmospheric pressure, humidity, temperature information or the like).

According to an embodiment, the applications 370 may include an application (hereinafter, referred to as “an information exchange application” for convenience of description) for supporting information exchange between the electronic device (for example, the electronic device 101) and an external electronic device (for example, the electronic device 102 or 104). The application associated with the exchange of information may include, for example, a notification relay application for transferring specific information to an external electronic device or a device management application for managing an external electronic device.

For example, the notification relay application may include a function of transferring, to the external electronic device (for example, the electronic device 102 or 104), notification information generated from the other applications of the electronic device (for example, the SMS/MMS application, the e-mail application, the health management application, and the environmental information application). Further, the notification relay application may receive notification information from, for example, the external electronic device and provide the received notification information to the user.

The device management application may manage (for example, install, delete, or update), for example, a function for at least a part of the external electronic device (for example, the electronic device 102 or 104) communicating with the electronic device (for example, turning on/off the external electronic device itself (or some elements thereof) or adjusting brightness (or resolution) of a display), applications executed in the external electronic device, or services provided from the external electronic device (for example, a telephone call service or a message service).

According to an embodiment, the applications 370 may include applications (for example, a health care application of a mobile medical appliance or the like) designated according to attributes of the external electronic device 102, 104 or 104. According to an embodiment, the applications 370 may include an application received from an external electronic device (for example, the server 106 or the electronic device 102o r 104). According to an embodiment, the application 370 may include a preloaded application or a third party application which can be downloaded from the server. The names of the components of the program module 310 according to the illustrated embodiment may vary according to the type of operating system.

According to various embodiments, at least a part of the programming module 310 may be implemented in software, firmware, hardware, or a combination of two or more thereof. At least some of the program module 310 may be implemented (for example, executed) by, for example, the processor (for example, the processor 210). At least some of the program module 310 may include, for example, a module, a program, a routine, a set of instructions, and/or a process for performing one or more functions.

The term “module” as used herein may, for example, mean a unit including one of hardware, software, and firmware or a combination of two or more of them. The “module” may be interchangeably used with, for example, the term “unit”, “logic”, “logical block”, “component”, or “circuit”. The “module” may be a minimum unit of an integrated component element or a part thereof. The “module” may be a minimum unit for performing one or more functions or a part thereof. The “module” may be mechanically or electronically implemented. For example, the “module” according to the present disclosure may include at least one of an Application-Specific Integrated Circuit (ASIC) chip, a Field-Programmable Gate Arrays (FPGA), and a programmable-logic device for performing operations which has been known or are to be developed hereinafter.

According to various embodiments, at least some of the devices (for example, modules or functions thereof) or the method (for example, operations) according to the present disclosure may be implemented by a command stored in a computer-readable storage medium in a programming module form. The instruction, when executed by a processor (e.g., the processor 120), may cause the one or more processors to execute the function corresponding to the instruction. The computer-readable storage medium may be, for example, the memory 130.

The computer readable recoding medium may include a hard disk, a floppy disk, magnetic media (e.g., a magnetic tape), optical media (e.g., a Compact Disc Read Only Memory (CD-ROM) and a Digital Versatile Disc (DVD)), magneto-optical media (e.g., a floptical disk), a hardware device (e.g., a Read Only Memory (ROM), a Random Access Memory (RAM), a flash memory), and the like. In addition, the program instructions may include high class language codes, which can be executed in a computer by using an interpreter, as well as machine codes made by a compiler. The aforementioned hardware device may be configured to operate as one or more software modules in order to perform the operation of the present disclosure, and vice versa.

The programming module according to the present disclosure may include one or more of the aforementioned components or may further include other additional components, or some of the aforementioned components may be omitted. Operations executed by a module, a programming module, or other component elements according to various embodiments of the present disclosure may be executed sequentially, in parallel, repeatedly, or in a heuristic manner. Further, some operations may be executed according to another order or may be omitted, or other operations may be added. Various embodiments disclosed herein are provided merely to easily describe technical details of the present disclosure and to help the understanding of the present disclosure, and are not intended to limit the present disclosure. Therefore, it should be construed that all modifications and changes or modified and changed forms based on the technical idea of the present disclosure fall within the present disclosure.

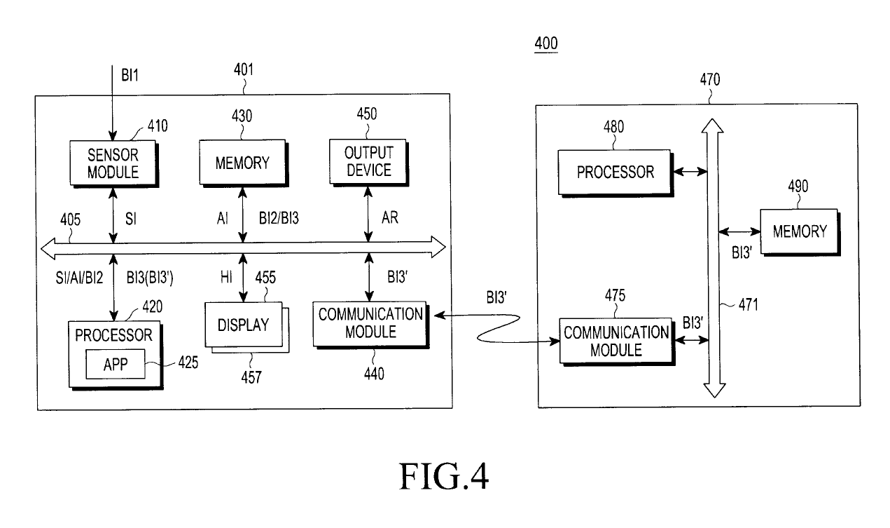

FIG. 4 is a schematic block diagram illustrating an electronic system according to an embodiment of the present disclosure.

In the following description, first biometric information (BI1) may refer to user's biometric information initially acquired through a sensor module. For example, the first biometric information (BI1) may refer to information on liquid or gas generated from the user such as user's tear, sweat, and exhalation.

Second biometric information BI2 may refer to user's biometric information corresponding to alcohol information (AI) of the first biometric information (BI1). For example, the second biometric information (BI2) may include user's biometric information related to alcohol in addition to user's alcohol consumption, calorie intake, nutrients, blood sugar, blood pressure, body fat percentage, body mass index, exercise quantity for calorie consumption, blood alcohol concentration, and/or heart rate.

Third biometric information (BI3 or BI3′) may refer to user's biometric information generated through accumulation of the alcohol information (AI) and the second biometric information (BI2). For example, the third biometric information (BI3 or BI3′) may include a value generated through accumulation of user's biometric information related to alcohol for a predetermined period in addition to user's alcohol consumption, calorie intake, nutrients, blood sugar, blood pressure, body fat percentage, body mass index, exercise quantity for calorie consumption, blood alcohol concentration, and/or heart rate.

Referring to FIG. 4, an electronic system 400 may include a first electronic device 401 and a second electronic device 470.

According to various embodiments, the electronic system 400 may acquire the user's first biometric information (BI1), analyze alcohol components included in the first biometric information (BI1), and acquire alcohol information (AI).

According to various embodiments, the electronic system 400 may determine a correlation between the alcohol information (AI) and the user's second biometric information (BI2) and provide health information (HI) to the user according to a result of the determination.

For example, the electronic system 400 may refer to a system that provides health information related to alcohol to the user by using the first electronic device 401.

The first electronic device 401 may acquire the alcohol information (AI) through the user's first biometric information (BI1) by using the sensor module 410, measure the user's second biometric information (BI2) corresponding to the alcohol information (AI), and provide user's health information (HI) by using the measured second biometric information (BI2).

For example, the first electronic device 401 may be implemented as a smart phone, a tablet PC, a mobile phone, a laptop PC, a desktop PC, a mobile medical device, a camera, or a wearable device.

The first electronic device 401 may include the sensor module 410, a processor 420, a memory 430, a communication module 440, an output device 450, and a display 455. Further, the elements of the first electronic device 401 may be connected through a bus 405.

The sensor module 410 may receive the user's first biometric information (BI1) and transmit sensing information (SI) to the processor 420.

The sensing information (SI) may refer to data (for example, component analysis data) on alcohol included in the first biometric information (BI1) analyzed by the sensor module 410. The alcohol may refer to an ethanol component.

The sensor module 410 may acquire the user's first biometric information (BI1). According to some embodiments, the sensor module 410 may acquire the first biometric information (BI1) from the user's skin or at least a part of the user's body.

According to some embodiments, the sensor module 410 may include an alcohol sensor (or an alcohol detection sensor). At this time, the alcohol sensor may be implemented as at least one of a semiconductor type sensor, an electrochemical type sensor, a catalyst type sensor, a calorie type sensor, a fluorescent type sensor, an infrared sensor, an optical sensor, a colorimetric sensor, and/or a conductive polymer-based sensor.

According to various embodiments, the processor 420 may detect the generation of an event related to alcohol through the sensor module 410.

According to some embodiments, the processor 420 may detect the user or a surrounding environment of the user through the sensor module 410 and detect the generation of the event related to alcohol according to a result of the detection.

The sensor module 410 may include a camera sensor, a temperature sensor, a voice sensor, and a pressure sensor.

The event may refer to some contextual event related to or otherwise associated alcohol. For example, the event may refer to a situation related to drinking such as an event related to alcoholic consumption, an atmosphere related to alcoholic consumption, a change in the situation during alcoholic consumption, and/or an alcoholic consumption state.

For example, the processor 420 may detect a user's drinking status by using at least one of a camera sensor (e.g., recognizing the user consuming an alcoholic beverage, or some visual indicator of a venue for consuming alcoholic beverages) and a sound sensor (e.g., recognizing audio cues indicating that alcohol is being consumed or certain noises indicating a venue where alcohol is consumed) included in the sensor module 410.

Meanwhile, the sensor module 410 may be implemented as substantially identical or similar to the sensor module 240 illustrated in FIG. 2.

The processor 420 may control the general operation of the first electronic device.

The processor 420 may receive sensing information (SI) from the sensor module 410. The processor 420 may generate (or measure) alcohol information (AI) within the user's body based on the sensing information (SI).

The processor 420 may generate second biometric information (BI2) corresponding to the alcohol information (AI) by using the alcohol information (AI).

According to some embodiments, when the first biometric information (BI1) is acquired through the sensor module 410, the processor 420 may analyze an alcohol component included in the first biometric information (BI1).

The processor 420 may measure the alcohol information (AI) by using the analyzed alcohol component.

The alcohol information (AI) may refer to information on an alcohol concentration within the user's body or a blood alcohol concentration of the user. For example, the alcohol information (AI) may include or be determined from alcohol type information, information on a user's gender, and information on a user's weight.

The alcohol type information may refer to information on the type of alcoholic beverage that was consumed by the user. For example, the alcohol type information may include information on various alcoholic beverages, including soju, beer, hard liquor, and/or grain wine, etc. The weight information may refer to information on a user's current or past weight. The gender information refers to information on a user's gender.

For example, the alcohol type information, the weight information, and/or the gender information may be preset to the first electronic device 401 by the user or a program.

According to various embodiments, the processor 420 may measure alcohol information (AI) based on the information on the type of alcohol which was consumed by the user, the user's gender information, and/or the user's weight information.

At this time, the processor 420 may measure an amount of alcohol which the user consumed through the measured alcohol information (AI) and equation (1).

c=A/(p*r) (1)

In equation (1), c denotes a blood alcohol concentration, A denotes an amount of alcohol which the user drank, p denotes a user's weight, and r denotes a coefficient according to a gender (male: r=0.7 and female r=0.6).

For example, the processor 420 may acquire the alcohol information (AI) based on the first biometric information (BI1) and acquire the user's blood alcohol concentration based on the alcohol information (AI). Further, the processor 420 may acquire the amount of alcohol which the user drank based on the blood alcohol concentration (c), the gender (r), and weight (p) information, which in this example, were preconfigured by the user.

The processor 420 may acquire alcohol consumption of the user based on equation (2).

A=alcohol consumption*alcohol concentration*0.7894 (2)

In equation (2), “A” denotes an amount of alcohol which the user consumed, e.g., indicating alcoholic consumption denoting a quantity of an alcohol beverage, and an “alcohol concentration” denotes an alcoholic concentration of an alcoholic beverage, which may be supplied or indicated according to alcohol type information.

According to some embodiments, the processor 420 may measure alcohol consumption based on equation (1) and equation (2). Further, the processor 420 may acquire second biometric information (BI2) based on the measured alcohol consumption.

The second biometric information (BI2) may refer to user's biometric information corresponding to the alcohol information (AI). For example, the second biometric information (BI2) may include a biometric change in the user caused by consumption of alcohol corresponding to the alcohol information (AI).

For example, when the second biometric information (BI2) corresponds to a user's blood pressure, a change in blood pressure (e.g., an increase or decrease) corresponding to the alcohol information (AI) may be included in the second biometric information (BI2).

The processor 420 may activate or deactivate the sensor module 410. According to some embodiments, the processor 420 may control the sensor module 410 to be activated periodically or aperiodically.

The processor 420 may facilitate manual and/or automatically activation or deactivation of the sensor module 410. For example, the processor 420 may control the sensor module 410 to periodically or aperiodically measure the user's first biometric information (BI1). For example, the processor 420 may control a measurement period of the user's first biometric information (BI1) through the sensor module 410.

For example, when a particular application is executed, the processor 420 may control the sensor module 410 to measure the first biometric information (BI1). Further, when a particular event (e.g., for example, an event related to alcohol consumption) is detected, the processor 420 may control the sensor module 410 to measure the first biometric information. Meanwhile, the processor 420 may control the first electronic device to allow the sensor module 410 to measure the first biometric information at a particular time (or particular period) and/or in a particular place. At this time, the particular event, the particular time, and/or the particular place may be set by the user.

The processor 420 may facilitate operation of an application 425. At this time, the application 425 may be executed by the processor 420.

For example, the application 425 may be an application related to health information, or an application that derives information from the sensor module 410.

The application 425 may control the first electronic device 401 to provide health information (HI) or an alarm signal (AR) to the user.

Hereinafter, for convenience of the description, an operation for controlling the first electronic device 401 by the application may be understood as an operation for controlling the first electronic device 401 by the processor 420.

The processor 420 may acquire the second biometric information (BI2) by using the alcohol information (AI). Further, the processor 420 may provide the health information (HI) to the user by using the second biometric information (BI2). Meanwhile, the processor 420 may provide the alarm signal (AR) to the user by using the second biometric information (BI2).

Further, the processor 420 may acquire (or measure) the second biometric information (BI2) regardless of the alcohol information (AI).

For example, the processor 420 may measure the user's second biometric information (BI2) in real time or periodically regardless of the alcohol information (AI) and, when the alcohol information (AI) is received, store second biometric information (BI2) corresponding to the received alcohol information (AI) among the measured second biometric information (BI2). At this time, when the alcohol information (AI) is received, the processor 420 may provide the health information (HI) and/or the alarm signal (AR) to the user by using the second biometric information (BI2) corresponding to the received alcohol information (AI).

According to some embodiments, the processor 420 may generate the health information (HI) based on the alcohol information (AI) and/or the second biometric information (BI2) and provide the health information (HI) to the user through the display 455. Further, the processor 420 may generate the alarm signal (AR) based on the alcohol information (AI) and/or the second biometric information (BI2) and provide an alarm to the user through the output device 450.

The processor 420 may compare the second biometric information (BI2) with a first reference value preset by the user and output the health information (HI) and/or the alarm signal (AR) according to a result of the comparison.

The health information (HI) may include information on user's health such as a correlation between the alcohol information (AI) and the second biometric information (BI2), information on user's health due to the drinking, drinking habit management, drinking habit improvement, second biometric information (BI2) management, healthy eating habit information, and/or nutrition information.

The alarm signal (AR) may refer to a signal that informs the user of whether the alcohol information (AI) and the second biometric information (BI2) have a correlation therebetween and/or whether the second biometric information (BI2) is in a dangerous level due to alcohol through the output device 450.

The first reference value refers to a reference value of the second biometric information (BI2) based on alcohol. For example, when the second biometric information (BI2) corresponds to alcohol consumption of the user, the user may set alcohol consumption (or alcohol intake) increased/decreased due to alcohol as the first reference value. For example, when a value of the alcohol consumption (or alcohol intake) due to alcohol exceeds the first reference value, the processor 420 may provide the health information (HI) and/or the alarm signal (AR) to the user.

Further, when the second biometric information (BI2) corresponds to a user's blood pressure, the user may set a value of the blood pressure increased due to alcohol as the first reference value. For example, when the value of the blood pressure increased/decreased due to alcohol exceeds the first reference value, the processor 420 may provide the health information (HI) and/or the alarm signal (AR) to the user.

The first reference value may be set by the user directly. Further, the first reference value may be automatically set by a program (or system) based on general health reference information.

The processor 420 may store the alcohol information (AI) and/or the second biometric information (BI2) in the memory 430.

Further, the processor 420 may read user's third biometric information (BI3), which has been accumulated and stored in the memory 430. For example, the user may set a period for which the third biometric information (BI3) is accumulated. For example, the user may set the third biometric information (BI3) to be accumulated in the unit of weeks, months, quarters, and years.

The processor 420 may determine a change in the third biometric information (BI3). For example, the processor 420 may determine third biometric information (BI3) corresponding to the alcohol information (AI) and determine a change in the third biometric information (BI3) corresponding to the alcohol information (AI).

According to various embodiments, the processor 420 may determine the third biometric information (BI3) due to alcohol which has been accumulated for a predetermined period, and the change in the third biometric information (BI3) due to alcohol.

Further, the processor 420 may generate the health information (HI) and/or the alarm signal (AR) based on the third biometric information (BI3) due to alcohol and the change in the third biometric information (BI3) due to alcohol.

According to some embodiments, the processor 420 may compare the third biometric information (BI3) with a second reference value preset by the user and output the health information (HI) and/or the alarm signal (AR) according to a result of the comparison. Further, the processor 420 may compare a change in the third biometric information (BI3) due to alcohol with the second reference value preset by the user and output the health information (HI) and/or the alarm signal (AR) according to a result of the comparison.

According to various embodiments, the processor 420 may receive third biometric information (BI3′) from the second electronic device 470 through the communication module 440. Further, the processor 420 may transmit the alcohol information (AI), the second biometric information (BI2), and/or the third biometric information (BI3) to the second electronic device 470 through the communication module 440.

The second reference value may refer to a reference value of the change in the third biometric information due to alcohol, which has been accumulated for a predetermined period.

According to some embodiments, when the third biometric information (BI3) corresponds to a blood pressure, the user may set a value of the blood pressure increased due to alcohol for the accumulation period as the second reference value. For example, when the value of the blood pressure increased/decreased due to alcohol for the accumulation period exceeds the second reference value, the processor 420 may provide the health information (HI) and/or the alarm signal (AR) to the user.

Further, when the third biometric information (BI3) corresponds to accumulated alcohol consumption, the user may set accumulated alcohol consumption (or accumulated alcohol intake) increased/decreased due to alcohol for the accumulation period as the second reference value. For example, when the value of the accumulated alcohol consumption (or accumulated alcohol intake) increased/decreased due to alcohol for the accumulation period exceeds the second reference value, the processor 420 may provide the health information (HI) and/or the alarm signal (AR) to the user.

The second reference value may be set by the user directly. Further, the second reference value may be automatically set by a program (or system) based on general health reference information.

The memory 430 may store the alcohol information (AI) and the second biometric information (BI2) generated by the processor 420. Further, the memory 430 may accumulate the alcohol information (AI) and the second biometric information for a predetermined period and store the accumulated information. At this time, the alcohol information (AI) and the second biometric information (BI2), which have been accumulated for the predetermined period, may be stored as the third biometric information (BI3).

According to some embodiments, the memory 430 may be implemented as a non-volatile memory. The memory 430 may be implemented to be substantially identical or similar to the memory 230 described in FIG. 2.

The communication module 440 may transmit and receive data to and from the second electronic device 470 through a wireless communication technology. The wireless communication technology may be implemented to be substantially identical or similar to the wireless communication technology described in FIGS. 1 to 3.

According to some embodiments, the communication module 440 may receive the third biometric information (BI3′) from the second electronic device 470. Further, the communication module 440 may transmit the alcohol information (AI), the second biometric information (BI2), and/or the third biometric information (BI3) to the second electronic device 470.

The output device 450 may provide an alarm (or alarm signal (AR)) to the user according to a control of the processor 420.

The output device 450 may include at least one of an optical output device, a speaker, a direction device, and a vibration device. For example, the output device 450 may provide the alarm (or alarm signal (AR)) to the user by using at least one of a light, a sound, a smell, and a vibration.

The display 455 may provide health information (HI) to the user according to a control of the processor 420. Further, the display 455 may provide the alarm to the user (or alarm signal (AR) according to a control of the processor 420.

According to various embodiments, the display 455 may include a touch screen 457. At this time, the user may input information on the user into the first electronic device 401 through the touch screen 457.

For example, the user may input biometric information on the user such as user's gender, user's weight, information on an alcohol type which the user drank, user's blood pressure, user's blood sugar, and/or user's heart rate through the touch screen 457.

The second electronic device 470 may receive the second biometric information (BI2) from the first electronic device 401 and store the second biometric information (BI2) as the third biometric information (BI3′) for a predetermined period. Further, the second electronic device 470 may transmit the third biometric information (BI3′) to the first electronic device 401. At this time, the second electronic device 470 may be implemented as the electronic device or server that stores the third biometric information (BI3′).

The second electronic device 470 may include a communication module 475, a processor 480, and a memory 490. The elements 475, 480, and 490 of the second electronic device 470 may be connected through the bus 471 and transmit data through the bus 471.

The communication module 475 may transmit the third biometric information (BI3′) to the first electronic device 401. Further, the communication module 475 may receive the second biometric information (BI2) and the alcohol information (AI) from the first electronic device 401.

The processor 480 may control the general operation of the second electronic device 470.

The memory 490 may store the second biometric information (BI2) transmitted from the communication module 475. Further, the memory 490 may store the second biometric information (BI2) as the third biometric information (BI3′) accumulated for a predetermined period.

The memory 490 may transmit the stored third biometric information (BI3′) to the communication module 475 according to a control of the processor 480.

The third biometric information (BI3′) may refer to the substantially same information as the third biometric information (BI3) stored in the first electronic device 401. However, unlike the third biometric information (BI3), the third biometric information (BI3′) may be stored in the second electronic device 470, and thus the storage locations thereof may be different.

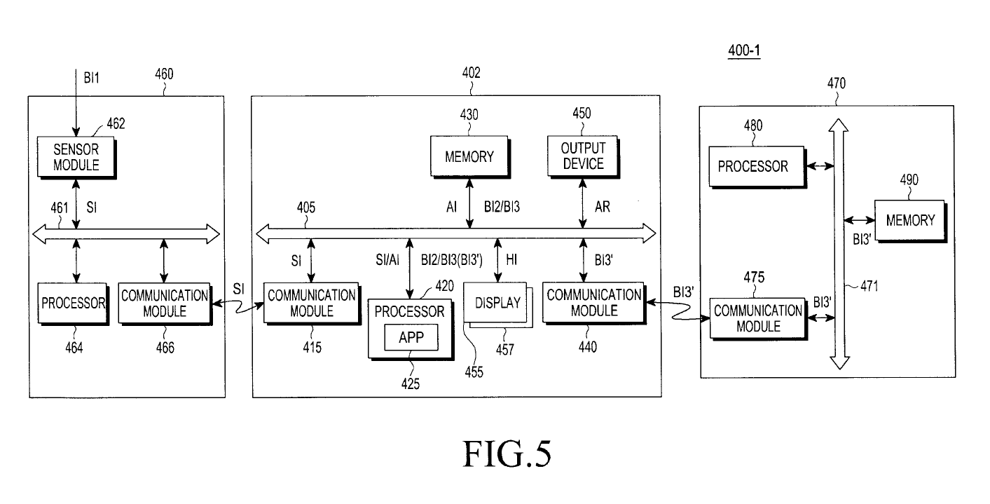

FIG. 5 is a schematic block diagram illustrating an electronic system according to another embodiment of the present disclosure.

Referring to FIGS. 4 and 5, an electronic system 400-1 may be implemented to be substantially identical or similar to the electronic system 400 illustrated in FIG. 4 except for a third electronic device 460.

A first electronic device 402 may be implemented to be substantially identical or similar to the first electronic device 401 illustrated in FIG. 4 except for the sensor module.

The first electronic device 402 may receive sensing information (SI) from the third electronic device 460 through a communication module 415.

The processor 420 may measure (or acquire) alcohol information (AI) based on the sensing information (SI) received from the third electronic device 460. Further, the processor 420 may acquire (or measure) second biometric information (BI2) corresponding to the alcohol information (AI).

The third electronic device 460 may receive user's first biometric information (BI1) and generate sensing information (SI) corresponding to the first biometric information (BI1). The first biometric information (BI1) and the sensing information (SI) may be substantially the same as that described in FIG. 4.

The third electronic device 460 may include a sensor module 462, a processor 464, and a communication module 466. The elements 462, 464, and 466 of the third electronic device 460 may be connected through a bus 461 and transmit and receive data through the bus 461.

For example, the third electronic device 460 may be implemented as a wearable device.

The sensor module 462 may receive the user's first biometric information (BI1). The sensor module 462 may analyze an alcohol component included in the first biometric information (BI1) and generate sensing information (SI). At this time, the sensing module 462 may be implemented to be substantially the same as the s module 410 described in FIG. 4.

The processor 464 may control the general operation of the third electronic device 460. The processor 464 may control activation or deactivation of the sensor module 462. For example, the processor 464 may activate the sensor module 462 in response to an event related to alcohol.

Further, the processor 464 may measure (or acquire) the first biometric information (BI1) periodically or aperiodically through the sensor module 462.

For example, the processor 464 may be implemented as a low power processor.

The communication module 466 may transmit the sensing information (SI) to the first electronic device 402 according to a control of the processor 464.

Although FIG. 5 illustrates that the first electronic device 402 does not include the sensor module and receives the sensing information (SI) corresponding to the first biometric information (BI1) from the third electronic device 460 for convenience of the description, the first electronic device 402 may be implemented to include the sensor module link the first electronic device 401 of FIG. 4 and to receive the sensing information (SI) from the third electronic device 460.

An electronic device according to various embodiments of the present disclosure may include a sensor, and a processor, wherein the processor may be configured to acquire first biometric information of a user through the sensor, to acquire alcohol information of the user based on the first biometric information, to acquire second biometric information of the user related to the alcohol information, and to provide health information to the user or another electronic device based on the second biometric information.

The processor may be configured to acquire the first biometric information based on at least one of preset functions related to alcohol.

The processor may be configured to acquire the first biometric information from a time point when at least one of the preset functions is executed to a time point when the function is terminated.

The processor may be configured to acquire at least one of tear, sweat, and exhalation of the user corresponding to the first biometric information.

The processor may be configured to provide the health information when the second biometric information is included in a predetermined range.

The processor may be configured to acquire at least one of a blood pressure, blood sugar, and heart rate corresponding to the second biometric information.

The electronic device may further include a communication module, such that the processor may be configured to acquire the second biometric information from an external device connected to the electronic device through the communication module.

The processor may be configured to determine a change in accumulated third biometric information of the user based on the second biometric information.

The processor may be configured to provide the health information based on a change in the third biometric information corresponding to the alcohol information among the change in the accumulated third biometric information.

The health information may include information on at least one of a blood pressure, blood sugar, alcohol level, alcohol consumption, a number of times the user drinks, calories, weight, and heart rate corresponding to the alcohol information.

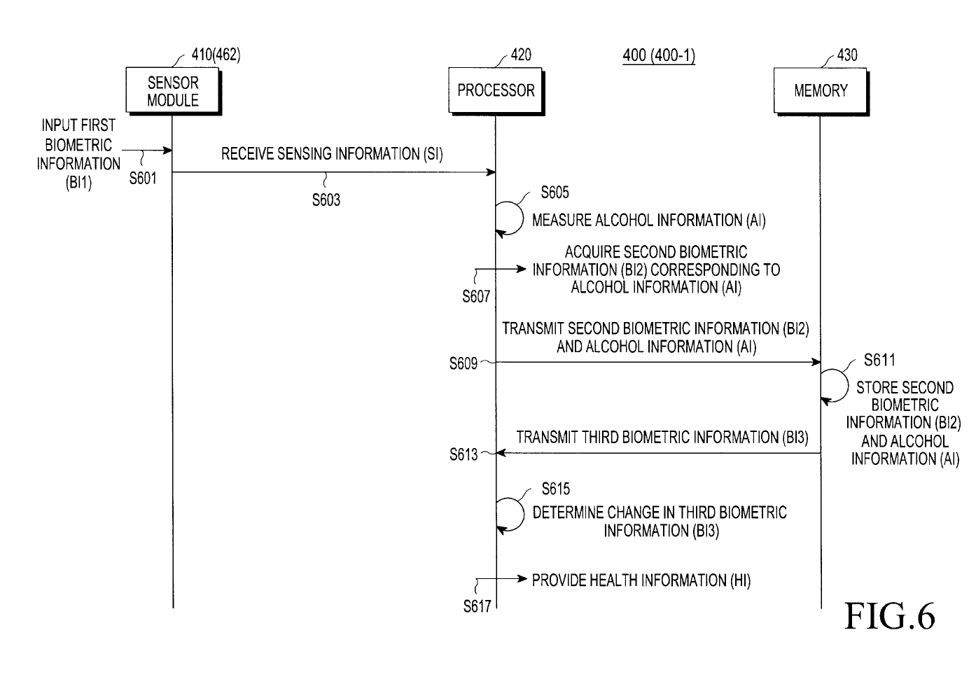

FIG. 6 is a data flow illustrating an operation method of an electronic device according to various embodiments of the present disclosure.

Referring to FIGS. 4 to 6, the electronic system 400 or 400-1 may provide health information (HI) or the alarm signal (AR) to the user in response to the first biometric information (BI1).

In operation S601, the first electronic device 401 (for example, the processor 420) may acquire the user's first biometric information (BI1) through the sensor module 410 or 462 (e.g., causing the biometric information to be ‘input,’ as depicted).

According to various embodiments, the first electronic device 401 may receive the first biometric information (BI1) via direct input to the sensor module 410 or 462. Alternatively, the first electronic device 401 may receive the first biometric information (BI1) by the sensor module 410 or 462 without the user being aware of the acquisition.

In operation S603, the processor 420 may acquire sensing information (SI) corresponding to the user's first biometric information (BI1) using the sensor module 410 or 462.

In operation S605, the processor 420 may measure (e.g., determine or acquire) alcohol information (AI) including a user's blood alcohol concentration (e.g., an alcohol concentration within the user's body) using the sensor information (SI).

In operation S607, the processor 420 may acquire user's second biometric information (BI2) corresponding to the alcohol information (AI). For example, the processor 420 may acquire blood pressure, blood sugar, heart rate, calories corresponding to the alcohol which was consumed, blood pressure changes (e.g., increase or decrease), weight, weight changes, and/or heart rate changes.

According to various embodiments, the processor 420 may compare the second biometric information with a first reference value, and provide health information (HI) or the alarm signal (AR) based on a result of the comparison.

For example, the processor 420 may measure blood pressure through the sensor module 410 and, when the measured blood pressure exceeds a predetermined reference value or a range, the processor 420 may provide certain health information (HI) or generate an alarm signal (AR).

In operation S609, the processor 420 may transmit the alcohol information (AI) and the second biometric information (BI2) to the memory 430.

In operation S611, the memory 430 may store the alcohol information (AI) and the second biometric information (BI2). According to various embodiments, the memory 430 may further store the second biometric information (BI2) in addition to the existing stored third biometric information (BI3). Further, the memory 430 may display the additionally stored second biometric information (BI2) based on the alcohol information (AI).

In some embodiments of the invention, the memory 430 may store third biometric information (BI3), which may include information that has been accumulated over a predetermined period of time.

In operation S613, the processor 420 may retrieve the accumulated and stored third biometric information (BI3) from the memory 430.

In operation S615, the processor 420 may determine a change in the third biometric information (BI3). For example, the third biometric information (BI3) may include information derived from or related to the second biometric information (BI2) due to the alcohol information (AI). For example, the processor 420 may determine biometric information corresponding to the alcohol information (AI) from the third biometric information (BI3).

In operation S617, the processor 420 may provide health information (HI) based on the third biometric information (BI3), and a change in the third biometric information (BI3). Further, the processor 420 may generate the alarm signal (AR) based on the third biometric information (BI3) and a change detected in the third biometric information (BI3).

According to various embodiments, the processor 420 may compare the third biometric information (BI3) with a second reference value and provide health information (HI) or the alarm signal (AR) according to a result of the comparison.

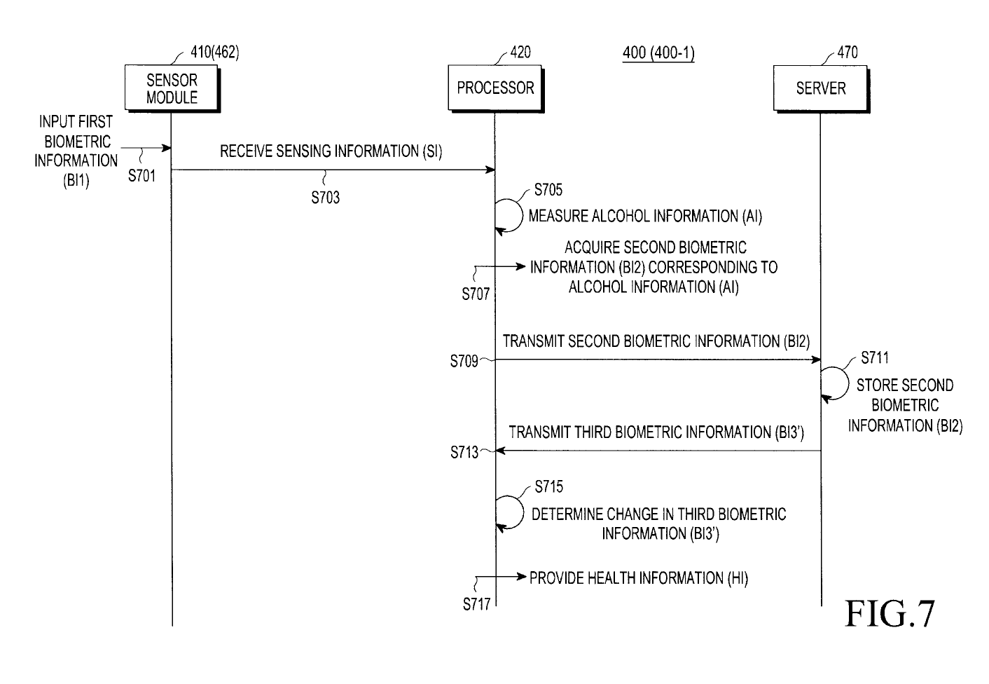

FIG. 7 is a data flow illustrating an operation method of an electronic device according to various embodiments of the present disclosure.

Referring to FIGS. 4 to 7, the electronic system 400 or 400-1 may provide health information (HI) or generate the alarm signal (AR) to notify the user in response to certain values of or within the first biometric information (BI1).

In operation S701, the first electronic device 401 (for example, the processor 420) may acquire user's first biometric information (BI1) through the sensor module 410 or 462.

According to various embodiments, the first electronic device 401 may directly receive the first biometric information (BI1) via the sensor module 410 or 462. Alternatively, the first electronic device 401 may receive the first biometric information (BI1) via the sensor module 410 or 462 without the user being made aware of the acquisition.

In operation S703, the processor 420 may acquire sensing information (SI) corresponding to the user's first biometric information (BI1) by using the sensor module 410 or 462.

In operation S705, the processor 420 may measure (or acquire) alcohol information (AI) including a user's blood alcohol concentration (or alcohol concentration within the body) using the sensor information (SI) corresponding to the first biometric information (BI1).

In operation S707, the processor 420 may acquire user's second biometric information corresponding to the alcohol information (AI). For example, the processor 420 may acquire a blood pressure, weight, heart rate, calories corresponding to alcohol which the user took, blood pressure change, weight change, and/or heart rate change.

According to various embodiments, the processor 420 may compare the second biometric information with a first reference value and provide health information (HI) or generated the alarm signal (AR) according to a result of the comparison. For example, the processor 420 may measure a blood pressure through the sensor module, and when the measured blood pressure exceeds a predetermined reference value or a predetermined range, the processor 420 may provide the health information (HI) or the alarm (AR).

In operation S709, the processor 420 may transmit the second biometric information (BI2) to the second electronic device 470. Further, the processor 420 may also transmit alcohol information (AI) to the second electronic device 470. In some embodiments, the second electronic device 470 may be implemented as a server.

In operation S711, the second electronic device 470 may store the second biometric information (BI2) in the memory 490. For example, the second electronic device 470 may further store the second biometric information (BI2) in addition to the preexisting (e.g., prestored) third biometric information (BI3′). Further, the second electronic device 470 may indicate (e.g., display) that the additionally stored second biometric information (BI2) is based on the alcohol information (AI).

The second electronic device 470 may store the third biometric information (BI3′), which may include biometric information accumulated over a predetermined period of time.

In operation S713, the processor 420 may receive the accumulated and stored third biometric information (BI3′) from the second electronic device 470.

In operation S715, the processor 420 may determine a change in the third biometric information (BI3′). For example, the third biometric information (BI3′) may include information derived from, indicated by, or informative on the second biometric information (BI2) based on the alcohol information (AI). The processor 420 may determine biometric information corresponding to the alcohol information (AI) among the third biometric information (BI3′).

In operation S717, the processor 420 may provide health information (HI) based on the third biometric information (BI3′) and the change in the third biometric information (BI3′). Further, the processor 420 may generate the alarm signal (AR) based on the third biometric information (BI3′) and the change in the third biometric information (BI3′).

According to various embodiments, the processor 420 may compare the change in the third biometric information (BI3) with a second reference value and provide health information (HI) or the alarm signal (AR) according to a result of the comparison.

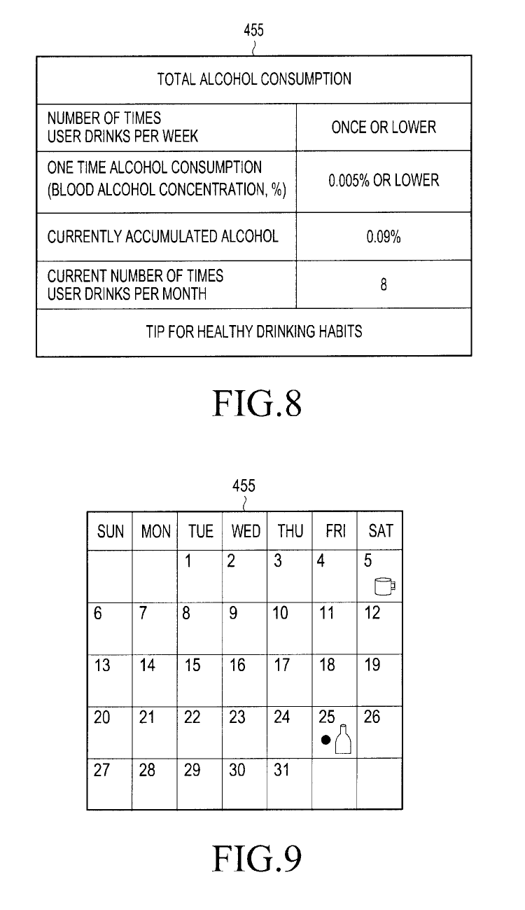

FIG. 8 illustrates an interface for health information provided by the electronic device according to an embodiment of the present disclosure.

Referring to FIGS. 4 to 8, the processor 420 may provide health information (HI) through the display 455. According to some embodiments, the processor 420 may provide alarm information on user's drinking information through the display 455.

The processor 420 may automatically generate a user's drinking history by using the alcohol information (AI), the second biometric information (BI2), and the third biometric information (BI3 or BI3′).

According to some embodiments, the processor 420 may provide information on user's total alcohol consumption, as measured for a predetermined period of time using the sensor module 410 or 462.

For example, the processor 420 may control display of a count (e.g., a number of times) the user consumes alcohol (e.g., drinks per week and/or per month). Further, the processor 420 may provide information on “one time” alcohol consumption (e.g., a present quantity consumed) and a blood alcohol concentration corresponding to “one time” alcohol consumption (e.g., a present blood alcohol level).

According to various embodiments, the processor 420 may provide an amount of alcohol consumed over a predetermined time. At this time, the predetermined period may be set by the user.

Further, the processor 420 may provide health information according to the count of the user's actual alcoholic drinks, and the user's alcohol consumption. For example, the processor 420 may determine a drinking pattern according to the number of times the user drinks alcoholic beverages and the user's alcohol consumption, and provide information that would modify this behavior, as relevant to a predetermined appropriate drinking habit, according to a result of the determination.

When the count of the user alcoholic drinks and/or the user's alcohol consumption exceeds a reference value set by the user, the processor 420 may generate the alarm signal (AR).

FIG. 9 illustrates an interface for health information provided by the electronic device according to another embodiment of the present disclosure.

Referring to FIG. 9, the processor 420 may provide health information (HI) through the display 455. According to some embodiments, the processor 420 may provide a drinking diary for display of a user's drinking record in a diary (e.g., calendar-based) format through the display 455.

The processor 420 may generate a user's consumption history using the alcohol information (AI), the second biometric information (BI2), and the third biometric information (BI3 or BI3′).

According to some embodiments, the processor 420 may record and display information on alcoholic consumption corresponding to a date when the alcohol information (AI) is measured through the sensor module 410 or 462.

For example, when the user drinks beer on the 5^{th} of the present month (as depicted in FIG. 9), the processor 420 may display an icon indicating consumption of alcohol (e.g., a special icon indicating beer was consumed) on the calendar area corresponding to the 5^{th}. Further, although not depicted in FIG. 9, the processor 420 may also display information on a time, a location, and/or a consumptive schedule in addition to the icon indicating alcoholic consumption (e.g., the beer icon).

In addition, when the user drinks soju on the 25^{th}, the processor 420 may display another icon indicating consumption soju on the area of the calendar corresponding to the 25^{th}. At this time, when an alcoholic intake of soju exceeds a first reference value (or second reference value), the processor 420 may additionally display a circle icon to indicate, for example, an excess of alcoholic consumption.

The information provided by the first electronic device illustrated in FIGS. 8 and 9 is only for convenience of the description and the technical idea of the present disclosure is not limited thereto.

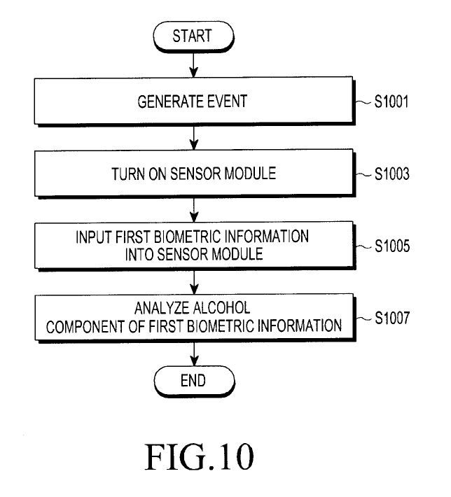

FIG. 10 is a flowchart illustrating an operation method of the electronic device according to various embodiments of the present disclosure.

Referring to FIG. 10, in operation S1001, the first electronic device 401 or 402 (for example, the processor 420) may identify an event (or preset function) for measuring a user's alcohol information (AI).

The event (or preset function) may be an event relevant to alcoholic consumption. However, in some embodiments, the event may also be irrelevant to alcoholic consumption. For example, the event may refer to a state (for example, a call state) in which an alcohol measurement application is executed, or where first biometric information (BI1) can be measured. In operation S1003, when an event is generated in the first electronic device, the processor 420 may activate the sensor module 410 or 462.

For example, when the event corresponds to execution of an alcohol concentration measurement application, the processor 420 may activate the sensor module 410 or 462 to measure an alcoholic concentration within the user's body.

According to various embodiments, when the event corresponds to execution of a phone call application, the processor 420 may activate the sensor module 410 or 462 to acquire user's exhalation (e.g., breath) information. Since the trigger is execution of the phone call application, this measurement is conducted in some embodiments without regard for a user's possible lack of alcoholic consumption. For example, the processor 420 may activate the sensor module 410 or 462 from the beginning of a phone call (e.g., execution of a phone call application) and terminate when the phone call terminates (e.g., termination of the phone call application). Further, the processor 420 may acquire a plurality of data for the exhalation information, ranging from a beginning of the executed phone call to the termination of the same.

Accordingly, when the sensor module 410 or 462 is activated, first biometric information (BI1) may be input into the sensor module 410 or 462 in operation S1005. For example, the first biometric information (BI1) may refer to user's breathing exhalation, or related biometric samples, such as liquids including saliva, sweat or tears.

For example, if the portable terminal is equipped with biometric sampling technology on the touch display, then when the display is touched, the processor 420 may acquire a user's biometric information according to the touch input (e.g., via sweat) through the sensor module 410 or 462. Further, the processor 420 may acquire user's tear information by using a contact type external device, such as a contact lens having biometric information collection and transmission functionality.

In operation S1007, the processor 420 may analyze (or measure) an alcohol component included in the first biometric information (BI1) using the sensor module 410 or 462 and output sensing information (SI). The sensor module 410 or 462 may transmit the sensing information (SI) to the processor 420 of the first electronic device 401 or 402.

According to various embodiments, the processor 420 may receive sensing information (SI) corresponding to the first biometric information (BI1) acquired through the sensor module 410 or 462 periodically or aperiodically.

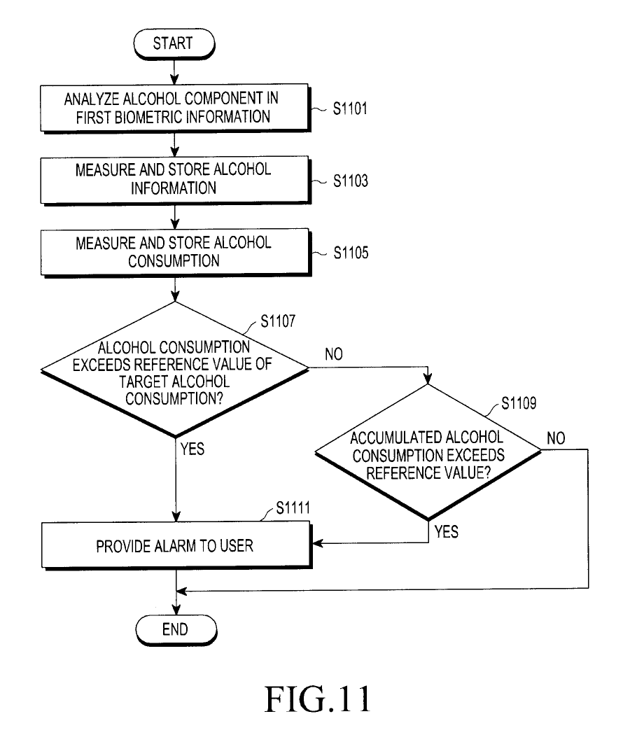

FIG. 11 is a flowchart illustrating an operation method of the electronic device according to various embodiments of the present disclosure.

Referring to FIG. 11, in operation S1101, the first electronic device 401 or 402 (for example, the processor 420) may measure an alcohol component of the first biometric information (BI1) using the sensor module 410 or 462 and generate sensing information (SI) on the measured alcohol component.

The sensor module 410 or 462 may transmit the sensing information (SI) to the processor 420.

In operation S1103, the processor 420 may measure alcohol information (AI) using the sensing information (SI), and store the alcohol information (AI) in the memory 430, and/or transmit the alcohol information (AI) to the second electronic device 470. For example, the first electronic device 401 or 402 may transmit the alcohol information (AI) to the server.

In operation S1105, the processor 420 may acquire (or measure) a user's alcoholic consumption using the alcohol information (AI). Further, the processor 420 may store the user's acquired alcoholic consumption in the memory 430 and/or transmit it to the second electronic device 470.

According to various embodiments, the processor 420 may store user's alcoholic consumption as part of alcohol information accumulated over a predetermined period of time in the memory 430. Further, the processor 420 may transmit the user's alcoholic consumption to the second electronic device 470 to store the user's alcohol consumption as measured over the predetermined period of the time in the second electronic device 470 (for example, a server).

According to various embodiments, the first electronic device 401 or 402 may acquire the user's alcoholic consumption through the second electronic device (for example, the server). For example, the first electronic device 401 or 402 may transmit user relevant information and alcoholic information (AI) to the server. The second electronic device 470 may transmit the user's alcoholic consumption (e.g., as aggregated/accumulated) based on the user information and the alcohol information (AI) to the first electronic device 401 or 402.

According to various embodiments, the user may set a target (e.g., a limitation) on alcoholic consumption by using the input device of the first electronic device 401 or 402. For example, the target alcohol consumption may refer to daily target alcohol consumption set by the user.

According to some embodiments, the user may set a first reference value of the target alcoholic consumption using a touch screen 457 of the first electronic device 401 or 402.

For example, the first electronic device 401 or 402 may acquire the first reference value of the target alcoholic consumption from the third electronic device 460 through the communication module 415.

According to various embodiments, the processor 420 may determine alcohol information (AI) resultant from the user's consumption, and measure user's alcoholic consumption corresponding to the alcohol information (AI).

In operation S1107, the processor 420 may determine whether the user's alcoholic consumption exceeds the first reference value indicating the target alcohol consumption.

When the user's alcohol consumption exceeds the first reference value of the target alcohol consumption in operation S1107, the processor 420 may provide the health information (HI) and/or generate the alarm signal (AR) to notify the user of excessive consumption in operation S1111.

Further, when the user's alcoholic consumption does not exceed the first reference value of the target alcoholic consumption, the processor 420 may determine whether alcoholic consumption accumulated for a predetermined period exceeds a second reference value in operation S1109.

When the alcoholic consumption accumulated for the predetermined period exceeds the second reference value in operation S1109, the processor 420 may provide the health information (HI) and/or generate the alarm signal (AR) to the user in operation S1111.

Further, when the alcoholic consumption accumulated for the predetermined period does not exceed the second reference value in operation S1109, the processor 420 may determine that the user's alcoholic consumption does not exceed the first reference value and the second reference value and does not provide the alarm signal (AR). For example, the processor 420 may periodically measure whether the user is consuming alcohol, and a quantity of the user's alcoholic consumption through the sensor module 410.

According to various embodiments, even though it is determined that the user's alcoholic consumption does not exceed the first reference value and the second reference value in operation S1109, the processor 420 may still provide information on the user's current alcoholic consumption to the user.

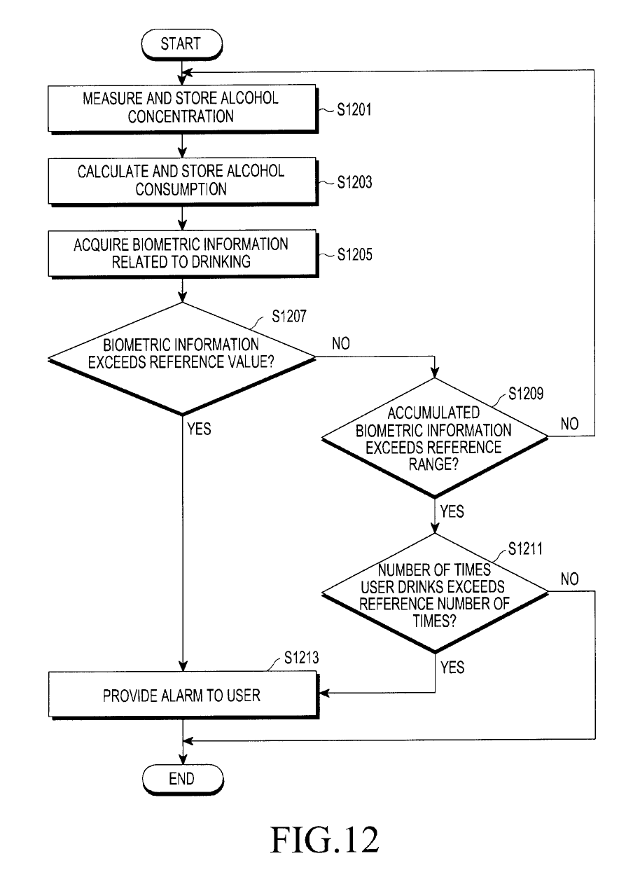

FIG. 12 is a flowchart illustrating an operation method of the electronic device according to various embodiments of the present disclosure.

Referring to FIG. 12, in operation S1201, the first electronic device 401 or 402 (for example, the processor 420) may measure alcohol information (AI) of the user's body in response to detected input of the user's first biometric information (BI1) via the sensor module 410 or 462. Further, the processor 420 may store the alcohol information (AI) in the memory 430. The processor 420 may transmit the alcohol information (AI) to the second electronic device 470.

In operation S1203, the processor 420 may measure (e.g., calculate) user's alcoholic consumption corresponding to the alcohol information (AI), and store the measured alcoholic consumption in the memory 430. Further, the processor 420 may transmit the alcohol information (AI) to the second electronic device 470.

In operation S1205, the processor 420 may acquire (or measure) second biometric information (BI2) corresponding to the alcohol information (AI). For example, the processor 420 may acquire user's biometric information such as change (e.g., an increase/decrease) in blood pressure, heart rate, caloric count or intake, and blood sugar resulting from the user's alcoholic consumption based on the alcohol information (AI). Further, the processor 420 may acquire information on the blood sugar, blood pressure, and heart rate related to the alcohol information (AI) through the sensor module 410.

In operation S1207, the processor 420 may compare the second biometric information (BI2) with the first reference value and determine whether the second biometric information (BI2) exceeds the first reference value. For example, the first reference value may indicate a limit or threshold of one or more values of the user's biometric information, including change in blood pressure, heart rate, caloric count, and blood sugar.

According to some embodiments, the processor 420 may determine whether the acquired second biometric information (BI2) exceeds the predetermined first reference value of a predetermined range. For example, when the acquired blood pressure information is 150 and the predetermined first reference value if 140, the processor 420 may determine that the user's blood pressure, as indicated in the second biometric information (BI2) exceeds the predetermined value.

According to various embodiments, the first reference value may be directly set by the user, or automatically set by the processor 420 based on biological information describing the user, such as gender and/or weight.

When the second biometric information (BI2) exceeds the first reference value, the processor 420 may provide the health information (HI) or generate the alarm signal (AR) to alert the user in operation S1213.

When the second biometric information (BI2) does not exceed the first reference value (S1207), the processor 420 may determine whether accumulated user's third biometric information (BI3 or BI3′) exceeds the second reference value in operation S1209.

When the accumulated third biometric information (BI3 or BI3′) does not exceed the second reference value in operation S1209, the processor 420 may continuously measure the user's alcohol information (AI) without separate or additional requests/provisions of biometric information. Meanwhile, the processor 420 may provide current alcoholic consumption information and/or alcohol information (AI) to the user.

When the accumulated third biometric information (BI3 or BI3′) exceeds the second reference value in S1209, the processor 420 may determine whether a count (e.g., the number of times) of the user's consumed alcoholic beverages for a designated period exceeds the reference count in operation S1211. For example, the reference count may be preset by the user or a program. Further, the reference count may be acquired through the second electronic device 470 (such as, for example, a server).

When the count of the user's consumed alcoholic beverages for the accumulation period exceeds the reference count, the processor 420 may provide the health information (HI) and/or generate the alarm signal (AR) to the user in operation S1213.

When the count of the user's consumed alcoholic beverages for the accumulation period does not exceed the reference count, the processor 420 may not provide information to the user. At this time, the processor 420 may continuously measure the alcohol information (AI) regarding the user's alcoholic consumption through the sensor module 410.

According to various embodiments, even though the count of the user's consumed alcoholic beverages for the accumulation period does not exceed the reference count, the processor 420 may separately provide current alcohol consumption or alcohol information (AI) to the user.

Further, even though the count of the user's consumed alcoholic beverages for the accumulation period does not exceed the reference number of times, the processor 420 may provide the alarm signal (AR) to the user. At this time, operation S1211 may be omitted.

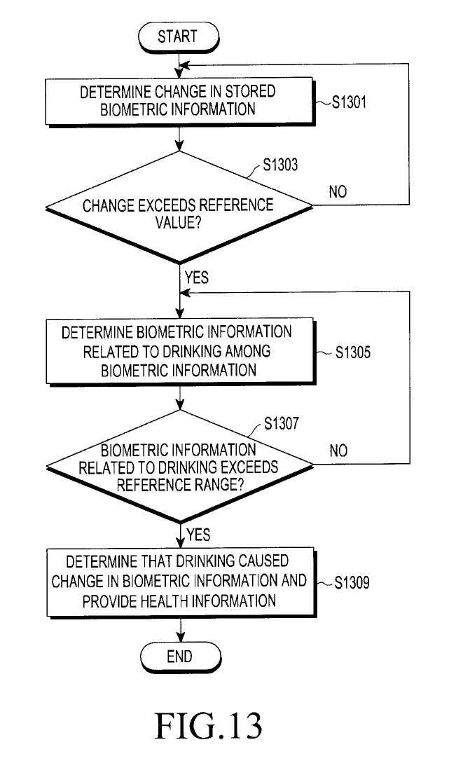

FIG. 13 is a flowchart illustrating an operation method of the electronic device according to various embodiments of the present disclosure.

Referring to FIG. 13, the first electronic device 401 or 402 (for example, the processor 420) may measure alcohol information (AI) indicating alcohol within the user's body in response to detecting input of the user's first biometric information (BI1) through the sensor module 410 or 462, and measure the second biometric information (BI2) corresponding to the alcohol information (AI).

The processor 420 may store the second biometric information (BI2) in the memory 430 or transmit the second biometric information (BI2) to the second electronic device 470. According to some embodiments, the processor 420 may acquire third biometric information (BI3 or BI3′) accumulated for a predetermined period based on the second biometric information (BI2).

In operation S1301, the processor 420 may determine a change in the third biometric information (BI3 or BI3′).

In operation S1303, the processor 420 may compare the change in the third biometric information (BI3 or BI3′) with a third reference value set by the user, and determine whether the change in the third biometric information (BI3) exceeds the third reference value.

The third reference value may refer to a reference value of the change in the accumulated user's third biometric information (BI3) set by the user, or automatically generated by the processor 420. For example, the third reference value may be set based on the user information, such as the user's gender or the user's weight.

When the change in the third biometric information (BI3 or BI3′) does not exceed the third reference value, the processor 420 does not provide information to the user and may continuously to monitor changes in the third biometric information (BI3 or BI3′).

When the change in the third biometric information (BI3 or BI3′) exceeds the third reference value, the processor 420 may determine biometric information related to the user's alcoholic consumption among the third biometric information (BI3 or BI3′) in operation S1305.

In operation S1307, the processor 420 may determine whether the biometric information related to the user's alcoholic consumption among the third biometric information (BI3 or BI3′) exceeds a fourth reference value (or reference range). The biometric information related to the user's alcoholic consumption may refer to biometric information corresponding to alcohol information (AI) for an accumulation period.

The fourth reference value may refer to a reference value of biometric information corresponding to the alcohol information (AI) among the accumulated user's third biometric information. At this time, the fourth reference value may be set by the user or automatically set by the processor 420.

When the biometric information related to alcoholic consumption among the third biometric information (BI3 or BI3′) does not exceed the reference value, the processor 420 does not provide information to the user and may continuously monitor the biometric information related to the user's alcoholic consumption among the third biometric information (BI3 or BI3′).

When the biometric information related to the drinking among the third biometric information (BI3 or BI3′) exceeds the reference value, the processor 420 may determine that the user's continued alcoholic consumption caused a change in the third biometric information (BI3 or BI3′) in operation S1309. Further, the processor 420 may provide the health information (HI) and/or generate the alarm signal (AR) related to the change in the third biometric information (BI3 or BI3′) to notify the user.

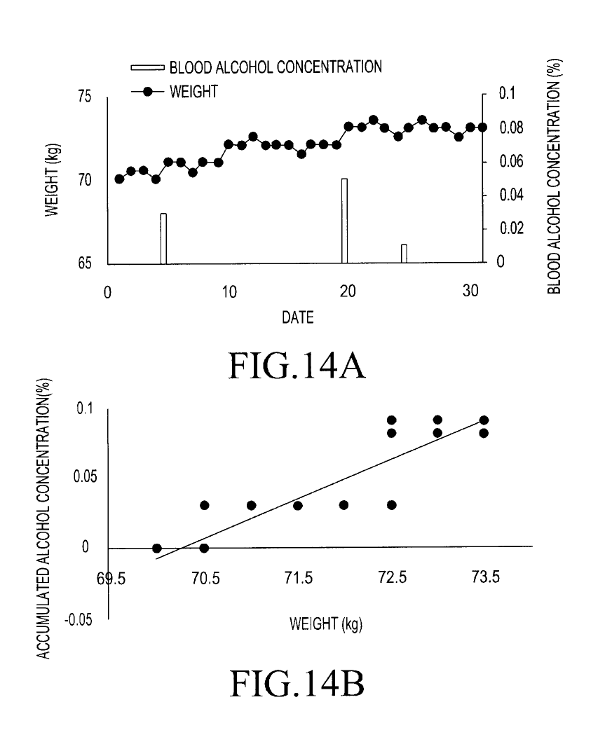

FIGS. 14A and 14B are graphs illustrating health information provided by the electronic device according to various embodiments of the present disclosure.

Referring to FIGS. 14A and 14B, the processor 420 may provide health information (HI) to the user through the display 455.

According to some embodiments, when the second biometric information (BI2) or the third biometric information (BI3 or BI3′) corresponds to the user's weight, the processor 420 may measure second biometric information (BI2) (for example, weight) corresponding to user's alcohol information (AI) (e.g., for example, blood alcohol concentration). Further, the processor 420 may provide information on a correlation between the user's drinking and weight.

FIGS. 14A-14B are graph illustrating the correlation between the user's drinking and the user's weight.

For example, in FIG. 14A, when the user's blood alcohol concentration increases due to drinking between the 0^{th} and the 10^{th} day, the processor 420 may provide a graph tracking the resulting gradual increase in the user's weight. Further, when the user's blood alcohol concentration increases due to drinking on the 20^{th} day, the processor 420 may provide a graph tracking the resulting continuously increase in the user's weight.

For example, the processor 420 may measure user's alcohol information (AI) across 30 days through the sensor module 410, and store second biometric information (BI2) (e.g., including weight) corresponding to the alcohol information (AI). Further, the processor 420 may thereby provide third biometric information (BI3 or BI3′) (for example, weight) for the accumulated 30 days in the format of a graph based on the second biometric information (BI2) (for example, weight), showing the resulting gradual increase in weight caused by the consumption of alcoholic beverages.

FIG. 14B is a graph illustrating the correlation between the user's drinking and the user's weight.

The processor 420 may store alcohol information (AI) tracking the user's drinking, and second biometric information (BI2) (indicating, for example, a user's weight) corresponding to the alcohol information (AI). Further, the processor 420 may provide the user's alcoholic consumption history for a particular monitoring period, along with resulting change in the user's weight in a graphical format.

For example, the processor 420 may provide a graph illustrating that the user's weight generally increases due to the user's drinking.

A method of operating an electronic device including a sensor may include an operation of acquiring first biometric information of a user through the sensor, an operation of acquiring alcohol information of the user based on the first biometric information, an operation of acquiring second biometric information of the user related to the alcohol information; and providing health information to the user or another electronic device based on the second biometric information.

The operation of acquiring the first biometric information may include an operation of acquiring the first biometric information based on at least one of preset functions related to alcohol.

The operation of providing the health information includes an operation of providing the health information when the second biometric information is included in a predetermined range.

The operation of acquiring the second biometric information may include an operation of acquiring the second biometric information from an external device connected to the electronic device through a wireless communication technology.

The method may further include an operation of determining a change in accumulated third biometric information of the user based on the second biometric information.

The operation of determining the change in the third biometric information may further include an operation of providing the health information based on a change in the third biometric information corresponding to the alcohol information among the change in the third biometric information.

The operation of providing the health information may include an operation of comparing the second biometric information with a preset first reference value and providing health information according to a result of the comparison.

The operation of providing the health information may include an operation of generating third biometric information accumulated for a preset period based on the second biometric information and comparing the third biometric information with a preset second reference value, and an operation of providing the health information according to a result of the comparison.

The operation of measuring the alcohol information may include an operation of detecting generation of an event related to alcohol through the sensor, and an operation of activating the sensor according to a result of the detection.

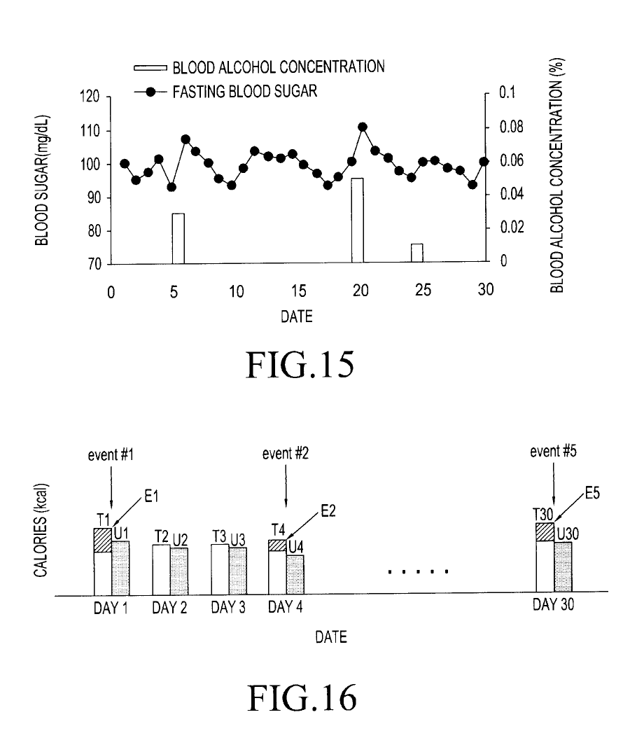

FIG. 15 is a graph illustrating health information provided by the electronic device according to various embodiments of the present disclosure.

Referring to FIG. 15, the processor 420 may provide health information (HI) to the user through the display 455.

According to some embodiments, when second biometric information (BI2) or third biometric information (BI3 or BI3′) corresponds to the user's blood sugar, the processor 420 may provide information illustrating a correlation between the user drinking and the blood sugar (or fasting blood sugar).

The processor 420 may measure second biometric information (BI2) (e.g., for example, fasting blood sugar) corresponding to user's alcohol information (AI) (for example, blood alcohol concentration). Further, the processor 420 may provide information on a correlation between the user's alcoholic consumption and the fasting blood sugar.

For example, the processor 420 may provide the information on the user's alcoholic consumption and the fasting blood sugar for the monitoring period in which data is accumulated in the form of a graph. For example, when the user's blood alcohol concentration increases, the processor 420 may provide a graph showing that the fasting blood sugar correspondingly increases (e.g., perhaps even instantaneously) to the user.

FIG. 16 is a graph illustrating health information provided by the electronic device according to various embodiments of the present disclosure.

Referring to FIG. 16, the processor 420 may provide health information (HI) to the user through the display 455.

According to some embodiments, when second biometric information (BI2) or third biometric information (BI3 or BI3′) corresponds to user's caloric intake, the processor 420 may provide information on a correlation between the user's alcoholic consumption and caloric intake.

The processor 420 may measure calories (e.g., E1 to E5) corresponding to user's alcohol information (AI). Further, the processor 420 may provide user's caloric intake (T1 to T30) and calories burnt (U1 to U30) to the user. At this time, the processor 420 may separately display the caloric intake (E1 to E5) corresponding to the alcohol information (AI) among the caloric intake (T1 to T30).

For example, the processor 420 may provide information on the user's caloric intake (T1 to T30) and calories burnt (U1 to U30) as monitored across 30 days in the form of a graph. At this time, the processor 420 may separately display the caloric intake (E1 to E5) due to the user's alcoholic consumption among the caloric intake (T1 to T30) to provide them to the user.

For example, the processor 420 may provide information on the caloric intake (T1) and the calories burnt (U1) on a first day (e.g., “day 1”) when the measurement for user's calories starts in the form of a graph. At this time, the processor 420 may separately display the caloric intake (E1) due to the user's alcoholic consumption on the graph.

Further, the processor 420 may also separately display generation of an event of the user's alcoholic consumption along with the graph. For example, the processor 420 may also display generation of the event of the user's alcoholic consumption on the first day (day 1) when the measurement of the user's calorie consumption starts along with the graph.

Accordingly, the processor 420 may receive information on calories implicated by the alcoholic consumption among the user's caloric intake (T1 to T30), so that there is a more dramatic indication of the correlation between the calories consumed and alcoholic consumption.

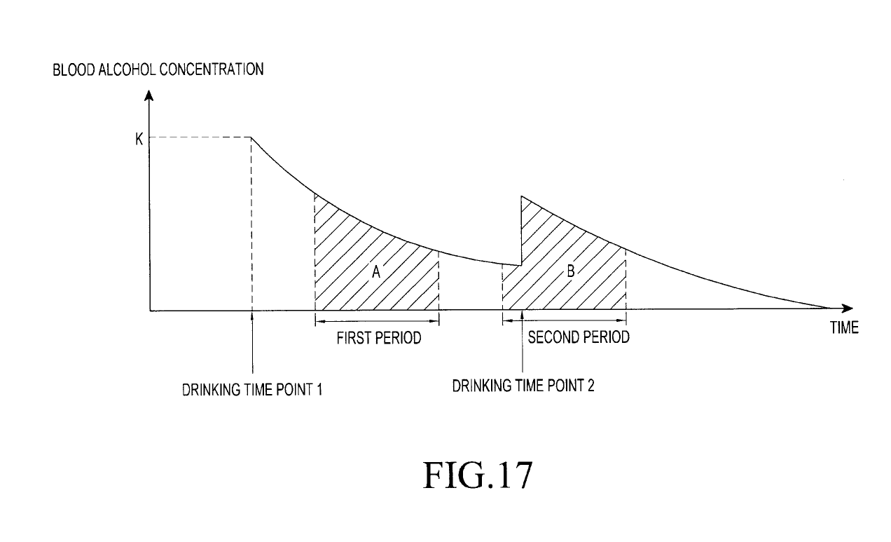

FIG. 17 is a graph illustrating a method of measuring health information by the electronic device according to an embodiment of the present disclosure.

Referring to FIG. 17, the processor 420 may measure alcohol information (AI) including a user's blood alcohol concentration in response to user's first biometric information (BI1) input through the sensor module 410 or 462.

The graph of FIG. 17 is a graph illustrating a change in the user's blood alcohol concentration over time (t).

At this time, a time point when the processor 420 measures the alcohol information (AI) may be different from a time point when the user consumes an alcoholic beverage. Further, the processor 420 may generate the time point when the user consumes alcohol after measuring the alcohol information (AI).

According to some embodiments, the processor 420 may measure the user's alcohol information (AI) during a first period (A). At this time, the processor 420 may measure the alcohol information (AI) after a first time point in which the user consumes alcohol.

Further, the processor 420 may acquire information on the first time point from the user. For example, the processor 420 may issue or display a request for information on the first time point to the user at a time when the alcohol information (AI) is measured. For example, the processor 420 may display a popup window (or popup screen) inquiring the user regarding the first time point through the touch screen and/or display 457. At this time, the processor 420 may determine the first time point based on information acquired from the user.

The processor 420 may measure the user's alcohol information (AI) to determine a decrease in (e.g., shown as a decreasing slope of) the user's blood alcohol concentration during the first period (A). For example, the processor 420 may measure the alcohol information (AI) including a user's maximum blood alcohol concentration, an alcoholytic time, and/or infer whether a “hangover” occurs based on the decrease in (or decreasing slope of) the user's blood alcohol concentration.

According to another embodiment, the processor 420 may measure the user's alcohol information (AI) during a second period (B). For example, the processor 420 may generate a user's second drinking time point after measuring the alcohol information (AI) when the user consumers a second alcoholic beverage.

The processor 420 may measure the user's alcohol information (AI) based on a decrease in (or a decreasing slope of) or an increase in (or an increasing slope of) the user's blood alcohol concentration during the second period (A). For example, the processor 420 may measure the alcohol information (AI) including a user's maximum blood alcohol concentration, an alcoholytic time, and/or infer whether a hangover occurs based on the decrease in (or the decreasing slope of) or the increase in (or the increasing slope of) the user's blood alcohol concentration.

Further, the processor 420 may measure the alcohol information (AI) based on at least one of a plurality of data of the user's first biometric information (BI1), which may be acquired during the first period (A) or the second period (B). For example, the processor 420 may acquire the biometric information such as a plurality of data received from the user's exhalation information, sweat, and tears, etc., and then measure the alcohol information (AI) from these samples using at least one of the data of acquired biometric information.

According to various embodiments of the present disclosure, a storage medium storing instructions is provided. The instructions are configured to instruct at least one processor to perform at least one operation when being instructed by the at least one processor. The at least one operation by an electronic device including a sensor may include an operation of acquiring first biometric information of a user through the sensor; an operation of acquiring alcohol information of the user based on the first biometric information; an operation of acquiring second biometric information of the user related to the alcohol information; and an operation of providing health information to the user or another electronic device based on the second biometric information.

Each of the components of the electronic device according to the present disclosure may be implemented by one or more components and the name of the corresponding component may vary depending on a type of the electronic device. In various embodiments, the electronic device may include at least one of the above-described elements. Some of the above-described elements may be omitted from the electronic device, or the electronic device may further include additional elements. Further, some of the components of the electronic device according to the various embodiments of the present disclosure may be combined to form a single entity, and thus, may equivalently execute functions of the corresponding elements prior to the combination.

The above-described embodiments of the present disclosure can be implemented in hardware, firmware or via the execution of software or computer code that can be stored in a recording medium such as a CD ROM, a Digital Versatile Disc (DVD), a magnetic tape, a RAM, a floppy disk, a hard disk, or a magneto-optical disk or computer code downloaded over a network originally stored on a remote recording medium or a non-transitory machine readable medium and to be stored on a local recording medium, so that the methods described herein can be rendered via such software that is stored on the recording medium using a general purpose computer, or a special processor or in programmable or dedicated hardware, such as an ASIC or FPGA. As would be understood in the art, the computer, the processor, microprocessor controller or the programmable hardware include memory components, e.g., RAM, ROM, Flash, etc. that may store or receive software or computer code that when accessed and executed by the computer, processor or hardware implement the processing methods described herein.

The control unit may include a microprocessor or any suitable type of processing circuitry, such as one or more general-purpose processors (e.g., ARM-based processors), a Digital Signal Processor (DSP), a Programmable Logic Device (PLD), an Application-Specific Integrated Circuit (ASIC), a Field-Programmable Gate Array (FPGA), a Graphical Processing Unit (GPU), a video card controller, etc.

In addition, it would be recognized that when a general purpose computer accesses code for implementing the processing shown herein, the execution of the code transforms the general purpose computer into a special purpose computer for executing the processing shown herein. Any of the functions and steps provided in the Figures may be implemented in hardware, software or a combination of both and may be performed in whole or in part within the programmed instructions of a computer. No claim element herein is to be construed under the provisions of 35 U.S.C. 112, sixth paragraph, unless the element is expressly recited using the phrase “means for”. In addition, an artisan understands and appreciates that a “processor” or “microprocessor” may be hardware in the claimed disclosure. Under the broadest reasonable interpretation, the appended claims are statutory subject matter in compliance with 35 U.S.C. §101.

What is claimed is:

1. An electronic device comprising:
a sensor; and
a processor configured to:
acquire, by the sensor, first biometric information of a user,
determine alcohol information for determining a presence of of alcohol in the user from the first biometric information,
determine second biometric information of the user related to the alcohol information, and
generate health information to be displayed to the user or transmitted to another electronic device based on the second biometric information.

2. The electronic device of claim 1, wherein the processor the first biometric information is acquired by the sensor in response to detecting occurrence of at least one preset function.

3. The electronic device of claim 2, wherein the processor is configured to acquire the first biometric information during a time range beginning from a first time point indicating a beginning of the occurrence of the at least one of the preset functions to a second time point indicating an end of the occurrence of the least one function.

4. The electronic device of claim 1, wherein the first biometric information is acquired when at least one of a tear, a droplet of sweat and an exhalation of the user contact the sensor.

5. The electronic device of claim 1, wherein the health information is generated in response to detecting that at least one value of the second biometric information is disposed within a predetermined value range.

6. The electronic device of claim 1, wherein the second biometric information includes information indicating at least one of blood pressure, blood sugar, and heart rate of the user.

7. The electronic device of claim 1, further comprising a communication module, wherein the processor is configured to acquire the second biometric information from an external device communicatively coupled to the electronic device through the communication module.

8. The electronic device of claim 1, wherein the processor is configured to retrieve third biometric information indicating at least one biometric value monitored for a predetermined time period, and determine whether a change in the at least one biometric value of the third biometric information has occurred based on the second biometric information.

9. The electronic device of claim 8, wherein the health information is generated in response to detecting the change in the at least one biometric value of the third biometric information,
wherein the change is determined as resulting from at least one value of the alcohol information.

10. The electronic device of claim 1, wherein the generated health information includes at least one of a blood pressure of the user, a blood sugar of the user, blood alcohol content level of the user, a total quantity of alcohol consumption, a count of alcoholic beverages consumed by the user, a total caloric intake of the user, a weight of the user, and a heart rate of the user.

11. A method in an electronic device including a sensor, comprising:
acquiring, by the sensor, first biometric information of a user through;
determining alcohol information for determining a presence of alcohol in the user from the first biometric information;
determining second biometric information of the user related to the alcohol information; and
generating health information to be displayed to the user or transmitted to another electronic device based on the second biometric information.

12. The method of claim 11, wherein the first biometric information is acquired by the sensor in response to detecting occurrence of at least one of preset function.

13. The method of claim 11, wherein the health information is generated in response to detecting that at least one value of the second biometric information is disposed within a predetermined value range.

14. The method of claim 11, wherein the acquiring of the second biometric information comprises acquiring the second biometric information from an external device communicatively coupled to the electronic device via wireless communication.

15. The method of claim 11, further comprising:
retrieving third biometric information indicating at least one biometric value monitored for a predetermined time period; and
determining a change in the at least one biometric value of the third biometric information based on the second biometric information.

16. The method of claim 15, wherein the the health information is generated in response to detecting the change in the at least one biometric value of the third biometric information,
wherein the change is determined as resulting from at least one value of the alcohol information.

17. The method of claim 11, wherein generating the health information comprises comparing the second biometric information with a preset first reference value and generating the health information according to a result of the comparison.

18. The method of claim 17, wherein generating the health information comprises:
generating third biometric information accumulated over a preset period of time based on the second biometric information and comparing the third biometric information with a preset second reference value; and
generating the health information according to a result of the comparison of the third biometric information with the preset second reference value.

19. The method of claim 11, wherein acquiring the alcohol information comprises:
in response to detecting, by the sensor, occurrence of an an event preconfigured as correlating to alcoholic consumption, activating the sensor to acquire the alcohol information.

20. A non-transitory storage medium storing instructions executable by at least one processor to perform at least one operation comprising:
acquiring, by the sensor, first biometric information of a user through;
determining alcohol information for determining a presence of alcohol in the user from the first biometric information;
determining second biometric information of the user related to the alcohol information; and
generating health information to be displayed to the user or transmitted to another electronic device based on the second biometric information.
